SEQUENCE LISTING (48 sequences)

SEQ ID NO: 1 - DNA, 5058 bp, Homo sapiens
gaagctctcctcgcggggcgggccggccggccgcacccccggcctggggcctccggtcgtagtaaagcgcaggcgggcggggaggcgggagcaggagcccgcggccggccagccgaagatggtggcggctactcctcctgtcatacacgtggacctaactgcaccagtagcttttctgagaatacttgctgaaaaggaagttttctggaatgggtaagtgtattctgattttcttgaacttttcttaaaaacaaatttttcttgctattaaagttgaataaataggattggtttcttagagagtaaaagtaggtgtttctttctttagacaatgtaccttttctgaaaaactaactcattaagtacggatttgctaattttaaggtagtaaaattacagtgtaaatattcctgtacatttttggaaactggcttatgcagtttacgaaatataattttagaccctcttttaagttgggtgataaagtagatataacctgagatgatagatttaaacaggatatttacgttctgctacaattgactgataacacttgaagtgtagtctgaacagtaattttgttaatcatttcaacaagtatttgctaagtggaagccagaagaggaggaaaatgttttgccacgtggatgtgaagatttcctctaaaaggtacacatggactaaattgcctttaaatgttccaaaattagttctcatttatttgcagtctcattttgttttgttttttttctctatgtgtcaatccatttgggagaggccagccattggaagagccaccacttccagtgctagttggatggttggttatgattgccttctgtaaagaattcttaaggcataaatacgtgtctaaatggacctcatatctttgagataattaaactaattttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcaaagtgcttacagtgcaggtagtgatatgtgcatctactgcagtgaaggcacttgtagcattatggtgacagctgcctcgggaagccaagttgggctttaaagtgcagggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaaagtgcttatagtgcaggtagtgtttagttatctactgcattatgagcacttaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgttgttagatttatttggctgttaagtattgcgatatgactaaacatactgtatacctgatgatcatctgtaaagttagagtatatctttttgctttctttggagttagtgttattccaggatattttacttaatctaaaagttaatttatgttgctcatatattactcaagtatttaaatttagagagaatgccgctctgtttaaagcaatgtgtaaagatgagtttttttaagcatggaatttagggttggggtacaatttgtttctattaagcaagtaccagtttaccaatacatgagtaactgaagtgtaactgttaaatgcttgtatactagtttttctttctgattgtcagtgatttataagctataaatgaccaaggtcctcagactgcttttagcatctgcaacttaaaaaaatgggagttagaaaaagaacaaatgctaaatagagtaacagttaaatgtatgtgtacactcttcccaaatgccaagagtgcagcggtggggtgagattcagatattcatttatttctaagtctgtagttaacatttatgttccctactccctacgtaagccagactttggcaacagtgatagttgattccaggcttatttgacttaaagtcactgaagtggaaactaagaagtggcagttagtgttttacccagcatttctgccttctctcttttcttcatgtgtttttgtctctagcctatgtgtatttgtgtagaataatgtgggatacctgaataatagatttaaaaggaccaagtggtaaaattgggcccaagctgaagtacaggcaaacttgatgtttgaaagataagttttgagaaatgtcattgtattttggagtaaaagaggctatcttagtaataagaaataaacttccataacactaggttagaccacccaataaatctagaaatcagcttttaaaaatattgtctgaagtctaacaaaagttttcacctctaatgtgttctttaagaaatttaaggaacttagccttggattcctgaatagaaaggtaagaattctatcattctggagttgatgaaaacataaattttcaggatgtgaaatgaacagtgatttataaaatggaaatcaaattgtacattagcagagttcttaagctttttgaattgaaggagacctaataattgtgtctttttggttatttagtgacaaacgtggctttcaaactatgcttaaaaagttccggctggacacggtggctcacacctataatcctagcacttggggaggctgaggcagacggattacctgaggtcaggagttcgagaccaacctggccgacatggtgaaacgctgtctctactaaaaatataaaaaattagccgggtgcagtggcgtgcacctgtaatcccagctactctggaggctgaggcaggagaatcacctgaacctgggaggtggaggtttcagtgagctgagatcctgccactgcactccagcctgggcgcaagaccaagacttaaacgcaaaaaaaaaaaaaaaaaaaaaaaaaaagtttcataatacagcatggtctggtagtttgcaaaatggtgtgcttttggggagatacactagcaatttttttaaaaactggaacagtgtgataggaagcctgctggatgatttcttaaatattctaaaatgtaagtcaaatatgttttaataacaaagacttaaatggcttttctccctagagactgaaactagtattcattgtgttcagaacttaattgggcttgaactgagatttaaatctaataaacaagttaataaatgtgtatgttttgttgtgggtttggtagtgatctgtggttctatagggtttaataggaattgcttttgatttgtttctggctttagaatgtgaggcaaattttacattcttggttctattaagattttcttaggcatgctaacatgccaacaaaaagccatgtaagtattgtataaaaagattcacattgttaatttagccattttgaaattcagatgagtgagcaagttgataatggcctcatctctgacctgagaaaaaacaactttgacccttgttcttaaaatgctttaaccttgaagttgcttgagacttaagaggtcatgttgctttaggtttaataaatagccttaactatttggaggggaaaaaatgggtcaacttttttttttttttttggcgtttgcatgtacaactttctatttttagcctatatttggaaagaaagcacttaacattttaggaattctttttaaagctgcttgcaaagtgttggtgattttactgaaaacttttgagatcttcattttacaggcagacctgtctaactacaagccagacttgggttttctcctgtagtttgaagacacactgactcctgacaaaatgcagcctgcaacttcctggagaacaactcagtgtcacattaaagtttattatgtatttaatgatacactgtttaattgacagttttgcatagtttgtctaactttagagaattaagagcctctcaactgagcagtaaaggtaaggagagctcaatctgcacagagccagtttttagtgtttgatggaaataagatcatcatgcccacttgagacttcagattattctttagcttagtggttgtatgagttacatcttattaaagtcgaaattaatgtagttttctgccttgataacatttcatatgtggtattagttttaaagggtcattaggaaaatgcacatattccatgaattttaagacccatagaaaagttgaagaatgcttaattttcttatccagtaatgtaaacacagagacagaacattgagatgtgcctagttctgtatttacagtttggtctggctgtttgagttctagcgcatttaatgttaataaataaaatactgcattttaaagctgttaagaaattgtccagaacgagaatattgaaataaaaacttcaaggttattatcgcagtttttatggcgtttttgttttgtcactac

SEQ ID NO: 2 - DNA, 84 bp, Homo sapiens
gtcagaataatgtcaaagtgcttacagtgcaggtagtgatatgtgcatctactgcagtgaaggcacttgtagcattatggtgac

SEQ ID NO: 3 - DNA, 71 bp, Homo sapiens
tgttctaaggtgcatctagtgcagatagtgaagtagattagcatctactgccctaagtgctccttctggca

SEQ ID NO: 4 - DNA, 82 bp, Homo sapiens
gcagtcctctgttagttttgcatagttgcactacaagaagaatgtagttgtgcaaatctatgcaaaactgatggtggcctgc

SEQ ID NO: 5 - DNA, 71 bp, Homo sapiens
gtagcactaaagtgcttatagtgcaggtagtgtttagttatctactgcattatgagcacttaaagtactgc

SEQ ID NO: 6 - DNA, 87 bp, Homo sapiens
cactgttctatggttagttttgcaggtttgcatccagctgtgtgatattctgctgtgcaaatccatgcaaaactgactgtggtagtg

SEQ ID NO: 7 - DNA, 78 bp, Homo sapiens
ctttctacacaggttgggatcggttgcaatgctgtgtttctgtatggtattgcacttgtcccggcctgttgagtttgg

SEQ ID NO: 8 - DNA, 81 bp, Homo sapiens
ccttggccatgtaaaagtgcttacagtgcaggtagctttttgagatctactgcaatgtaagcacttcttacattaccatgg

SEQ ID NO: 9 - DNA, 71 bp, Homo sapiens
tgtgttaaggtgcatctagtgcagttagtgaagcagcttagaatctactgccctaaatgccccttctggca

SEQ ID NO: 10 - DNA, 96 bp, Homo sapiens
acattgctacttacaattagttttgcaggtttgcatttcagcgtatatatgtatatgtggctgtgcaaatccatgcaaaactgattgtgataatgt

SEQ ID NO: 11 - DNA, 69 bp, Homo sapiens
agtaccaaagtgctcatagtgcaggtagttttggcatgactctactgtagtatgggcacttccagtact

SEQ ID NO: 12 - DNA, 75 bp, Homo sapiens
tcatccctgggtggggatttgttgcattacttgtgttctatataaagtattgcacttgtcccggcctgtggaaga

SEQ ID NO: 13 - DNA, 75 bp, Homo sapiens
tgttgtcgggtggatcacgatgcaattttgatgagtatcataggagaaaaattgcacggtatccatctgtaaacc

SEQ ID NO: 14 - DNA, 82 bp, Homo sapiens
cctgccggggctaaagtgctgacagtgcagatagtggtcctctccgtgctaccgcactgtgggtacttgctgctccagcagg

SEQ ID NO: 15 - DNA, 80 bp, Homo sapiens
ctgggggctccaaagtgctgttcgtgcaggtagtgtgattacccaacctactgctgagctagcacttcccgagcccccgg

SEQ ID NO: 16 - DNA, 84 bp, Homo sapiens
ggccagtgttgagaggcggagacttgggcaattgctggacgctgccctgggcattgcacttgtctcggtctgacagtgccggcc

SEQ ID NO: 17 - DNA, 23 bp, Homo sapiens
caaagtgcttacagtgcaggtag

SEQ ID NO: 18 - DNA, 22 bp, Homo sapiens
actgcagtgaaggcacttgtag

SEQ ID NO: 19 - DNA, 23 bp, Homo sapiens
taaggtgcatctagtgcagatag

SEQ ID NO: 20 - DNA, 23 bp, Homo sapiens
actgccctaagtgctccttctgg

SEQ ID NO: 21 - DNA, 22 bp, Homo sapiens
agttttgcatagttgcactaca

SEQ ID NO: 22 - DNA, 23 bp, Homo sapiens
tgtgcaaatctatgcaaaactga

SEQ ID NO: 23 - DNA, 23 bp, Homo sapiens
taaagtgcttatagtgcaggtag

SEQ ID NO: 24 - DNA, 22 bp, Homo sapiens
actgcattatgagcacttaaag

SEQ ID NO: 25 - DNA, 23 bp, Homo sapiens
agttttgcaggtttgcatccagc

SEQ ID NO: 26 - DNA, 23 bp, Homo sapiens
tgtgcaaatccatgcaaaactga

SEQ ID NO: 27 - DNA, 23 bp, Homo sapiens
aggttgggatcggttgcaatgct

SEQ ID NO: 28 - DNA, 22 bp, Homo sapiens
tattgcacttgtcccggcctgt

SEQ ID NO: 29 - DNA, 23 bp, Homo sapiens
aaaagtgcttacagtgcaggtag

SEQ ID NO: 30 - DNA, 22 bp, Homo sapiens
ctgcaatgtaagcacttcttac

SEQ ID NO: 31 - DNA, 23 bp, Homo sapiens
taaggtgcatctagtgcagttag

SEQ ID NO: 32 - DNA, 22 bp, Homo sapiens
tgccctaaatgccccttctggc

SEQ ID NO: 33 - DNA, 22 bp, Homo sapiens
agttttgcaggtttgcatttca

SEQ ID NO: 34 - DNA, 23 bp, Homo sapiens
tgtgcaaatccatgcaaaactga

SEQ ID NO: 35 - DNA, 23 bp, Homo sapiens
caaagtgctcatagtgcaggtag

SEQ ID NO: 36 - DNA, 22 bp, Homo sapiens
actgtagtatgggcacttccag

SEQ ID NO: 37 - DNA, 22 bp, Homo sapiens
gggtggggatttgttgcattac

SEQ ID NO: 38 - DNA, 22 bp, Homo sapiens
tattgcacttgtcccggcctgt

SEQ ID NO: 39 - DNA, 22 bp, Homo sapiens
cgggtggatcacgatgcaattt

SEQ ID NO: 40 - DNA, 22 bp, Homo sapiens
aattgcacggtatccatctgta

SEQ ID NO: 41 - DNA, 21 bp, Homo sapiens
taaagtgctgacagtgcagat

SEQ ID NO: 42 - DNA, 22 bp, Homo sapiens
ccgcactgtgggtacttgctgc

SEQ ID NO: 43 - DNA, 23 bp, Homo sapiens
caaagtgctgttcgtgcaggtag

SEQ ID NO: 44 - DNA, 22 bp, Homo sapiens
actgctgagctagcacttcccg

SEQ ID NO: 45 - DNA, 21 bp, Homo sapiens
aggcggagacttgggcaattg

SEQ ID NO: 46 - DNA, 22 bp, Homo sapiens
cattgcacttgtctcggtctga

SEQ ID NO: 47 - DNA, 20 bp, Artificial sequence
  Synthetic crRNA Rcan3 #1119
gagaaatacgaactgcacgc

SEQ ID NO: 48 - DNA, 20 bp, artificial sequence
  Synthetic crRNA Rcan3 #1558
gatggtcttcggtgaaaatg

(19) United States
(12) Patent Application Publication
(10) Pub. No.: US 2022/0177882 A1
(43) Pub. Date: Jun. 9, 2022

(54) CALCINEURIN INHIBITOR RESISTANT IMMUNE CELLS FOR USE IN ADOPTIVE CELL TRANSFER THERAPY

(71) Applicant: UNIVERSITÄT BASEL, Basel (CH)

(72) Inventors: Lukas JEKER, Riehen (CH); Marianne DÖLZ, Allschwil (CH)

(21) Appl. No.: 17/426,385

(22) PCT Filed: Jan. 31, 2020
(86) PCT No.: PCT/EP2020/052454

§ 371 Date: Jul. 28, 2021

(30) Foreign Application Priority Data
Feb. 1, 2019 (EP) 19155132.4

(51) Int. Cl. C12N 15/113; A61K 35/17; A61P 35/00; C12N 15/87

(52) U.S. Cl. CPC C12N 15/113; C12N 15/87; A61P 35/00; A61K 35/17

(57) ABSTRACT

The present invention relates to immune cells in which the regulatory activity of miR-17˜92 cluster or paralogs thereof is increased to confer calcineurin inhibitor resistance. In particular said immune cell is engineered to overexpress at least one mi RNA of miR-17˜92 cluster or paralogs thereof or to inactivate at least one miR-17˜92 cluster target gene to confer calcineurin inhibitor resistance. Particularly, the present invention relates to the use of calcineurin inhibitor-resistant immune cells in combination with calcineurin inhibitor in adoptive cell transfer therapy in a patient in need thereof.

### FIELD OF THE INVENTION

The present invention relates to immune cells in which the regulatory activity of miR-17˜92 cluster or paralogs thereof is increased to confer calcineurin inhibitor resistance. In particular said immune cell is engineered to overexpress at least one miRNA of miR-17˜92 cluster or paralogs thereof or to inactivate at least one miR-17˜92 cluster target gene to confer calcineurin inhibitor resistance. Particularly, the present invention relates to the use of calcineurin inhibitor-resistant immune cells in combination with calcineurin inhibitor in adoptive cell transfer therapy in a patient in need thereof.

### BACKGROUND OF THE INVENTION

CD4 T cells which are essential for the adaptive immune response differentiate into different T cell subsets characterized by the expression of distinct transcription factors and cytokines. CD4 T cells also provide help to B cells to generate germinal center (GC) responses. The activation of CD4 T cells is strongly dependent on T cell receptor (TCR), which acts synergistically with co-stimulatory receptor CD28. Similarly, CD8 T cells and other types of T cells depend on activation through their TCR in combination with costimulatory molecules. Signalling via CD28 is important for proliferation and expansion (Levine, B. L., et al. J Immunol, 1997. 159(12):5921-30), but also to regulate IL-2 production (Sanchez-Lockhart, M., et al. J Immunol, 2004. 173(12):7120-4). Furthermore, CD28 expression is essential for the glycolytic switch during T cell activation (Frauwirth, K. A., et al. Immunity, 2002. 16(6):769-77; Jacobs, S. R., et al. J Immunol, 2008. 180(7):4476-86). Apart from priming, CD28 stimulation is required for the differentiation and maintenance of T-cell helper type 1 (TH1) as well as follicular helper T (TFH) cells during response to viral infection (Linterman, M. A., et al. Elife, 2014. 3) and for the formation of a GC response (Ferguson, S. E., et al. J Immunol, 1996. 156(12):4576-81). Nevertheless, even if extensive studies have been realised on the function of CD28 (Esensten, J. H., et al. Immunity, 2016. 44(5):973-88), a complete understanding of the pathways initiated by this receptor is still missing.

During T cell activation, while overall RNA transcription is increased, miRNAs are globally down regulated (Bronevetsky, Y., et al. J Exp Med, 2013. 210(2):417-32). Transcription of miRNAs results in a long primary transcript (pri-miRNA) (Lee, Y., et al. EMBO J, 2004. 23(20):4051-60), which gets processed by Drosha and DGCR8 in the nucleus (pre-miRNA) (Gregory, R. I., et al. Nature, 2004. 432(7014):235-40). This pre-miRNA is exported to the cytosol where the RNAse III endonuclease Dicer processes it into double stranded RNA duplices, which are the miRNA and its antisense strand (Hutvagner, G., et al. Science, 2001. 293(5531):834-8). The mature miRNA is then able to guide the RNA induced silencing complex (RISC) to its target mRNA, a process that is mainly determined by the seed region of the miRNA that binds to the target gene 3′UTR (Bartel, D. P. Cell, 2004. 116(2):281-97; Kim, V. N. Nat Rev Mol Cell Biol, 2005. 6(5):376-85). Upon targeting of the mRNA, the protein expression is usually down regulated by mRNA cleavage, mRNA decay or translational repression (Baek, D., et al. Nature, 2008. 455(7209):64-71; Selbach, M., et al. Nature, 2008. 455(7209):58-63).

In contrast to the global miRNA down regulation, expression of miR-17˜92 and the two paralog clusters miR-106a˜363 and miR-106b˜25 is maintained or even upregulated during T cell activation. MiR-17˜92 is induced upon CD28 co-stimulation (de Kouchkovsky, D., et al. J Immunol, 2013. 191(4):1594-605). The miR-17˜92 cluster codes for six microRNAs (miR-17, miR-18a, miR-19a, miR-20a, miR-19b, and miR-92) which are classified into groups according to their four seed families (Xiao, C. and K. Rajewsky. Cell, 2009. 136(1):26-36) (miR17 family, miR18 family, miR19 family and miR92 family). Functionally, miR-17˜92 is important for proliferation (Jiang, S., et al. Blood, 2011. 118(20):5487-97), but also differentiation into various T helper subsets including TFH (Baumjohann, D. Cancer Lett, 2018. 423:147-152; Baumjohann, D., et al. Nat Immunol, 2013. 14(8):840-8; Jeker, L. T. and J. A. Bluestone. Immunol Rev, 2013. 253(1):65-81). Even if many targets of this miRNA cluster, e.g. PTEN (Xiao, C., et al. Nat Immunol, 2008. 9(4):405-14) but also KLF-2 (Serr, I., et al. Proc Natl Acad Sci USA, 2016. 113(43):E6659-E6668) and Rorα (Baumjohann, D., et al. Nat Immunol, 2013. 14(8):840-8), have been reported and validated so far, none of these targets could fully explain the phenotypes observed when the cluster expression is altered. This suggests that the sum of many targets that effect small changes might be needed to change the cell fate (Jeker, L. T. and J. A. Immunol Rev, 2013. 253(1):65-81). The expression of miR-17˜92 is important for aspects of T cell activation that are also influenced by CD28 expression, like proliferation (Baumjohann, D., et al. Nat Immunol, 2013. 14(8):840-8; Xiao, C., et al. Nat Immunol, 2008. 9(4):405-14), metabolism (Izreig, S., et al. Cell Rep, 2016. 16(7):1915-28) and the differentiation of TFH and GC B cells (Baumjohann, D., et al. Nat Immunol, 2013. 14(8):840-8).

Adoptive cell transfer, which involves the transfer of immune cells, is a promising strategy to treat viral infections, autoimmune disease and cancer. Using principles of synthetic biology, advances in immunology and genetic engineering have made it possible to generate human T-cells that display desired specificities and enhanced functionalities.

In immunocompetent patients, transplanted cells or organs are rapidly rejected by the host immune system if they originate from a different donor individual than the recipient. Immunologically, such cells are called allogeneic and the immunologic rejection is called allogeneic rejection. The exceptions are transplantations between genetically identical twins. Thus, to prevent rejection of the allogeneic graft, the subject's immune system must be suppressed. Immunosuppressive agents that target different pathways of the immune system are widely used therapeutically for immunosuppression. One example is targeting calcineurin by Cyclosporin A (CsA) or FK506 (Tacrolimus), which prevents the activation of T cells. Another drug, CTLA-4-Ig prevents activation of CD28 costimulation and therefore similarly suppresses T cell activation. However, in the case of adoptive cell transfer (ACT) therapy, the use of immune suppressive agent may have a detrimental effect on the grafted immune cells. Therefore, it remains a need to develop immune cells resistant to the immunosuppressive agent treatment, to effectively use an adoptive immunotherapy approach in these conditions.

### SUMMARY OF THE INVENTION

Using CD4cre.miR1792tg.CD28ko mice, the inventors showed that transgenic expression of miR-17˜92 can compensate CD28 signalling in vitro and in vivo and correct the transcriptome of CD28ko cells. Additionally, the inventors provide evidence that miR-17˜92 promotes calcineurin/NFAT activity, NFAT nuclear translocation and drives an NFAT-dependent gene expression signature. Moreover, the inventors demonstrate that miR-17˜92 cluster targets Rcan3, a repressor of the calcineurin-NFAT axis and they furthermore demonstrate that the decreased expression of this biological calcineurin inhibitor at the same time conveys the cells with a higher resistance to a chemical calcineurin inhibitor. The inventors also demonstrate that forced miR-17˜92 expression not only restores costimulatory function in CD28-deficient T cells and imposes CNI resistance in those cells but also conveys CNI resistance to wildtype, i.e. CD28-sufficient T cells. They also showed that the inactivation of a miR-17˜92 cluster target gene, Rcan3 gene conveys CNI resistance to immune cells.

The present invention relates to a calcineurin inhibitor (CNI)-resistant immune cell in which regulatory activity, preferably expression of the miR-17˜92 cluster or paralogs thereof is increased or miR-17˜92 target genes decreased for use in adoptive cell transfer therapy in a subject in need thereof wherein said immune cell is administered in said subject in combination with a CNI.

In a particular embodiment, said immune cell is engineered to overexpress at least one miRNA selected from the group consisting of miR-17, miR-18a, miR-19a, miR-20a, miR-19b-1, miR-92a-1, miR-106a, miR-18b, miR-19b-2, miR-20b, miR-92a-2, miR363, miR-106b, miR-93, miR-25, preferably miR-17 or miR-19. In a preferred embodiment said immune cell is engineered by introducing into said immune cell a nucleic acid construct comprising at least one miRNA sequence selected from the group consisting of: SEQ ID NO: 17 to 46, more preferably at least one pre-miRNA sequence selected from the group consisting of: SEQ ID NO: 1 to 16. In a more preferred embodiment, said nucleic acid construct is introduced by electroporation.

In another particular embodiment, said immune cell is engineered to inactivate or repress the expression of at least one miR-17˜92 cluster target gene, preferably Rcan3 gene preferably said immune cell is engineered by introducing a Cas9/CRISPR complex able to target Rcan3 gene into said immune cell.

In a particular embodiment, said calcineurin inhibitor is selected form the group consisting of: cyclosporine A, FK506 and CTLA-4 Ig.

In another particular embodiment, the immune cell is selected from the group consisting of: T cell, B cell, tumor infiltrating lymphocytes, NK cell, macrophage and regulatory T cell and originate from said subject or a donor. In a preferred embodiment, said immune cell further express a recombinant antigen receptor, preferably a chimeric antigen receptor.

In a particular embodiment, the adoptive cell transfer therapy is used for the treatment of a cancer, an autoimmune disease, an inflammatory disease, an infection, a disease requiring hematopoietic stem cells transplantation (HSCT) or the prevention of organ rejection, preferably a disease selected from the group consisting of: graft versus host disease, hematologic malignancy and posttransplant lymphoproliferative disease. The present invention also relates to a pharmaceutical composition comprising a CNI-resistant immune cell as previously described and a calcineurin inhibitor, in particular for use in adoptive cell transfer therapy in a subject in need thereof.

### BRIEF DESCRIPTION OF THE DRAWINGS

FIG. 1. miR-17˜92 deficiency phenocopies CD28-deficiency. miR1792lox (grey, left), wt (black), miR1792tg (grey, right A) Quantification of flow cytometry intracellular IL-2 staining in CD4⁺ cells stimulated for 3 h with PMA/Iono/BFA; B) IL-2 secretion measured by ELISA in culture supernatants at 48 h; C) Proliferation of CFSE labelled T^{1792Δ/Δ} (grey, left), wt (black), and T^{1792tg/tg} (grey, right) CD4⁺ T cells activated for 48 h with plate-bound αCD3/αCD28 mAbs. Error bars show means±SD, Dunn's multiple comparison test, p values: ns=not significant, *<0.05 **<0.002 ***<0.0002 ****<0.0001. Data represent 2-3 independent experiments with 3-4 biological replicates per group.

FIG. 2. miR-17˜92 expression modifies metabolism in activated CD4 T cells. CD4′T cells from T^{1791Δ/Δ} (light grey), wt (black), T^{1792/tg/tg} (grey) were assessed for their metabolic activity by mitochondria stress test with seahorse machine. A) Mitochondria stress test measured with a 96-well seahorse in naïve CD4⁺ T cells. Two experiments with 3-4 biological replicates per group and experiment are shown. left: Extracellular acidification rate, right: Oxygen consumption rate. Both parameters are overlapping in the three genotypes. B) Basal respiration and ATP coupled respiration in naïve CD4⁺ T cells. C) Mitochondria stress test measured in CD4⁺ T cells activated for 48 h and D) Basal respiration and ATP coupled respiration in activated CD4⁺ T cells. Pooled 3-8 biological replicates from 4 experiments are shown. Tukey's multiple comparison test, p values: *<0.05 E) RNA sequencing data shows an enrichment of genes associated with TCA. Shown is gene set REACTOME_TCA_CYCLE_AND_RESPIRATORY_ELECTRON_TRANSPORT enrichment with genes differentially expressed between T^{1792Δ/Δ} and T^{1792tg/tg} in naïve, 24 h and 48 h post activation. Colors indicate fold change direction with grey left being upregulated in T^{1792Δ/Δ} and grey right downregulated.

FIG. 3. Transgenic miR-17˜92 expression restores costimulation of CD28 deficient T-cells in vitro. wt (black), CD28^{-/-} (dark grey, middle), CD28^{-/-} with miR1792tg (=rescue) (light grey, right). A) Quantification of flow cytometry intracellular IL-2 staining in CD4⁺ T cells stimulated for 3 h with PMA/Iono/BFA. B) Proliferation measured by CFSE dilution, gated on viable CD4⁺ T cells. Representative histograms of each genotype activated without (blank) or with (grey) αCD28. C) Blasting of CD4⁺ T cells shown as MFI of FSC-A of the lymphocyte gate. D-E) CD4⁺ T cells were stimulated for 48 h with plate-bound αCD3 with (+) or without (−) αCD28 and investigated for expression of early activation markers CD25/CD69 expression (D) as well as CD44/CD62L expression (E). Data from 3 independent experiments with 3-4 biological replicates per group.

Error bars represent mean±SD, Tukey's or Hom-Sidak's multiple comparison test; p values: ns=not significant, *<0.05 **<0.002 ***<0.0002 ****<0.0001.

FIG. 4. Transgenic miR-17˜92 expression can partially compensate for CD28 signal during in vitro differentiation. Naïve CD4 T cells were activated for 24 h, 48 h, and 72 h with plate-bound antibodies (0.2 μg/ml αCD28, 0.5 μg/ml αCD3) in the presence of skewing conditions for the generation of TH1 (50 U IL-2, 5 ng/ml IL12 and 10 μg/ml αIL4 per ml T cell medium), TH17 (50 ng/ml IL6, 3 ng/ml TGFβ, 5 μg/ml αIFNγ and 10 μg/ml αIL4 per ml T cell medium) and iTreg (250 U IL-2, 0.75 ng/ml TGFβ, 10 μg/ml αIFNγ and 10 μg/ml αIL4 plus 0.9 mM retinoic acid). wt (black, left), CD28ko (dark grey, second from left), rescue (grey, second from right) and miR1792tg (light grey, right). Data from two independent experiments are shown, with representative FACS plots of each time point and genotype. A) TH1 differentiation was stained with IFNγ and Tbx21 (Tbet) in viable CD4 T cells. The artificial expression of miR-17˜92 forces IFNγ production. B) TH17 were stained for IL-17A and Rorγt in viable CD4 T cells, Rorγt⁺ are the two top quadrants while Rorγt⁺IL17A⁺ cells are only the top right quadrant. C) iTregs were stained with CD25 and Foxp3, data summary shows % FoxP3⁺CD25⁺ population.

FIG. 5. Transgenic miR-17˜92 expression restores costimulation of CD28-deficient cells in vivo. 6-8 week old mice were infected with LCMV Armstrong; spleens were analyzed at d8 post infection. wt (black), CD28^{-/-} (dark grey), rescue (light grey). A-D) represent data from 4 independent experiments with 4 mice per group, pre-gating on viable CD4⁺CD3⁺ or viable CD19⁺B220⁺ cells. A) CD44 expression. B) relative number of Bcl6⁺ICOS⁺ population (TFH). C) relative number of CXCR5⁺PD-1⁺ population (TFH). D) relative number of Fas⁺GL7⁺ population (GC B cells). E) representative contour plots of Tbx21 and IFNγ expression. F) Quantification of Tbx21⁺IFNγ⁺ CD3⁺CD4⁺ cells. G) ratio of Tbx21⁺IFNγ⁺ to total Tbx21⁺ cells. Error bars represent mean with SD, Dunn's multiple comparison test; p values: ns=not significant, *<0.05, **<0.002, ***<0.0002, ****<0.0001.

FIG. 6. Loss of miR-17˜92 expression phenocopies CD28 deficiency in vivo during LCMV infection, and heterozygous expression of miR-17˜92 can partially rescue CD28ko. 6-8 week old mice were infected with 2*10⁵ PFU LCMV Armstrong i.p. and spleens were analyzed at day 8 post infection like in FIG. 5. wt (left), T^{1792Δ/Δ} (second from left) CD28^{-/-} (third from left), CD28^{-/-} with heterozygous transgenic miR-17˜92 expression=hetrescue (third from right), CD28^{-/-} with transgenic miR-17˜92 expression=rescue (second from right), T^{1792tg/tg} (right). A-D) represent data from four independent experiments with 3-4 mice per group gated on viable CD4⁺CD3⁺ or viable CD19⁺B220⁺ cells. A) CD44 expression B) quantification of % Bcl6⁺ICOS⁺ (TFH) C) % CXCR5⁺PD-1⁺ (TFH) and D) % Fas⁺GL7⁺ (GC B cells). Error bars represent mean with SD, Dunn's multiple comparison test, p values: ns=not significant, *<0.0332, **<0.0021, ***<0.0002, ****<0.0001. E-G) Splenocytes were restimulated with GP-64 and BFA for 4 h and investigated for TH1 phenotype, pre-gated on viable CD3+CD4+ cells. Shown are 2-3 independent experiments with 3-4 biological replicates per group. E) representative contour plots of Tbx21 and IFNγ expression. F) Tbx21⁺IFNγ⁺ data summary. G) ratio of Tbx21⁺IFNγ⁺ to total Tbx21⁺ cells.

FIG. 7. Restoration of CD28 function by miR-17˜92 is cell intrinsic. Adoptive transfer of naïve SMARTA⁺ CD4⁺ T cells into CD28^{-/-} hosts, subsequent LCMV Armstrong infection and analysis of organs at d8 post infection. Donor genotypes wt (black, left), CD28^{-/-} (grey, middle), rescue (grey, right). Dotted line indicates recipient intrinsic Vα2⁺Vβ8.3⁺ population measured in a non-transferred control host. A) Vα2⁺Vβ8.3⁺ cells from peripheral lymph node (LN) pre-gated on viable, CD3⁺CD4⁺ cells; B) CD44 expression in Vα2⁺Vβ8.3⁺ population from peripheral LN. 2 independent experiments, 4 recipients per group. Error bars represent mean±SD, Dunn's multiple comparison test, p values: ns=not significant, *<0.05, **<0.002, ***<0.0002. C-D) Vα2⁺Vβ8.3⁺ cells in viable CD4⁺ population from spleen (C) and mesenteric LN (D); E-F) CD44 expression in Vα2+Vβ8.3+ population from spleen (E) and mesenteric LN (F). 2 independent experiments, 4 recipients per group. Error bars represent mean±SD, Dunn's multiple comparison test, p values: ns=not significant, *<0.05, **<0.002, ***<0.0002.

FIG. 8 miR-17˜92 shapes the transcriptome and promotes NFAT-dependent gene expression after T cell activation. Naïve CD4+ T cells from T^{1792Δ/Δ} (light grey), T^{1792tg/tg} (grey) or wt (black) were activated with plate-bound αCD28 and αCD3 for 0, 24 h and 48 h. Total RNA was extracted for bulk RNA sequencing. A) PCA (PC1 vs. PC2) based on the 25% most variable genes. B) Hierarchical clustering of set of genes selected with abs(log 2FC)>1 & adj.P.Val<0.001 in the T^{1792tg/tg} vs. T^{1792Δ/Δ} comparison at 24 h. The heatmap displays the centered log of counts per million. Annotations “DE” indicate the fold change direction, “DE intron” indicates if there is significant changes are observed in EISA analysis, “TS” indicates presence (grey) or absence (blank) of a seed match and its location and “AHC” indicates the 3′UTR signal intensity in HITS-CLIP data. Boxes I-IVb designate gene clusters. C) Volcano plot showing the absolute log 2 Fold change and −log 10 pvalue from regulon analysis. A threshold of 1% FDR was applied. Dot size indicates number of genes within each regulon and colors indicate fold change direction. D) Heatmap of genes under NFATC2_D and NFATC3_D regulons plus several known activated genes in CD4 cells (I110, I112a, 116, Rorc, Il23a). Hierarchical clustering was applied on genes. Centered log of counts per million is displayed. E-F) Genome-wide transcriptome analysis, presented as the log 2 value of the gene-expression ratio for each gene vs. the cumulative fraction of all log 2 ratios in naïve (E) and 24 h activated (F). Shown is the miR-17 seed family for T^{1792tg/tg} vs. wt and T^{1792Δ/Δ} vs. comparisons. Black curve: all genes of the data set that do not have a seed match and showed five or less AHC reads, grey: subset of genes that has a seed sequence for the seed family and >5 reads in the AHC.

FIG. 9. miR-17˜92 expression partially rescues the transcriptome in CD28ko cells. CD4⁺ T cells from T^{1792Δ/Δ} (grey), wt (black), T^{1792tg/tg} (light grey), CD28^{-/-} (dark grey) and rescue (grey) mice were activated for 24 h. Total RNA was extracted for sequencing. A) PCA (PC1 vs. PC2) based on the 25% most variable genes of the dataset B) Genome-wide transcriptome analysis, presented as the log 2 value of the gene-expression ratio for each gene versus the cumulative fraction of all log 2 ratios. Shown are the contrasts between activated samples separated by the miR-17 seed family for the comparison CD28^{-/-} vs. wt and rescue vs. wt. Black curve: genes that do not have a seed match, <5 AHC reads and no differential expression in the second RNA sequencing. Grey: genes with a conserved binding site for the miR-17 seed family (TS), >5 AHC reads and no differential expression in the AHC, grey: genes with a conserved binding site for the miR-17 seed family (TS) and >5 reads in the AHC and differential expression in the second RNA sequencing data set.

FIG. 10. Rcan3 expression and Cyclosporin A sensitivity is modified by miR-17˜92 and CD28 expression. A) Binding sites of Argonaute 2 in 3′UTR of Rcan3 detected by HITS-CLIP and the predicted miR-17 target site indicated indicated by the flag. B) Rcan3 mRNA expression detected with qPCR 24 h after activation, shown are pooled data from three independent experiments normalized to wt. T^{1792Δ/Δ} (left), wt (black), T^{1792tg/tg} (right). mRNA expression was normalized to 18S ribosomal RNA. Values are means±SD, Dunn's multiple comparison test, p values: ns=not significant, *<0.05, ****<0.0001. C) Rcan3 protein abundance measured by targeted proteomics 24 h after activation. CD28^{-/-} (left), T^{1792Δ/Δ} (second from left), rescue (middle), and T^{1792tg/tg} (right) are compared to wt (second from right). Proteins were isolated from the same cells for targeted proteomics of Rcan3 and total RNA sequencing. Numbers indicate p values from T-test. D) CD4⁺ wt (black), CD28^{-/-} (dark grey) and rescue (light grey) cells were activated for 48 h in the presence of increasing concentrations of Cyclosporin A (CsA) as indicated and stained for CD25 and CD69. Left: representative plots of CD25/CD69 expression in viable CD4 T cells activated for 48 h with no or 6.25 ng/ml CsA. Right: percentage of the CD25+CD69+ population as gated on the left. Shown are two independent experiments, error bars represent means±SD. Tukey's multiple comparison, p values: **<0.002, ****<0.0001 refer to the difference between CD28^{-/-} and wt. E) influence of 6.25 ng/ml CsA on blasting (FSC-A of the lymphocyte gate) of viable CD4+ cells. F) Imagestream analysis of CD4⁺ T cells activated for 48 h in presence of 6.25 ng/ml Cyclosporin A, stained for DAPI and NFATc2. Examples for cytoplasmic (top) and nuclear (bottom) NFATc2 in a CD28^{-/-} sample. G) Histograms of the similarity dilate, indicative of the co-localization of NFATc2 and DAPI signal, gates indicate the translocated population (high similarity dilate) and the cytoplasmic population (low similarity dilate).

FIG. 11: Transgenic miR-17˜92 expression in wt CD4+ and CD8+ T cells confers the cells with a higher resistance to calcineurin inhibitors (CsA and FK506). A, B) CD4 T cells from miR1792tg (grey) or wt (black) mice were activated for 48 h in the presence of increasing concentrations of Cyclosporin A (CsA) as indicated (A) or FK506 (B) and stained for their expression of CD25 and CD69. Shown are two independent experiments, error bars represent means±SD. Holm-Sidaks's multiple comparison, p values: ns>0.1234 *<0.0332 **<0.0021, ***<0.0002 ****<0.0001 refer to the difference between miR1792tg and wt. C,D) CD8+ T cells from miR1792tg (grey) or wt (black) mice were activated for 48 h in the presence of increasing concentrations of CsA (C) or FK506 (D) as indicated and stained for their expression of CD25 and CD69. Shown are two independent experiments, error bars represent means±SD. Holm-Sidaks's multiple comparison, p values: ns>0.1234 *<0.0332 **<0.0021, ***<0.0002 ****<0.0001 refer to the difference between miR1792tg and wt.

FIG. 12: CRISPR/Cas9-mediated Rcan3 deletion, a miR-17˜92 target gene, confers resistance to CsA

CD4 T cells electroporated with control gRNA or gRNA targeting Rcan3 (CIC domain or exon 2 (crRNA 1119, crRNA1558)) were activated 48 h in the presence of increasing concentrations of CsA as indicated. CD44 expression as a marker of T cell activation was quantified by flow cytometry. MFT: Mean fluorescence intensity.

### DETAILED DESCRIPTION OF THE INVENTION

In immunocompetent patients, (adoptively) transferred organs or cells are rapidly rejected by the host immune system. In addition, in the context of allogeneic HSCT, the grafted immune system can attack the host causing a disease called graft versus host diseases (GvHD). To prevent graft complication, immunosuppressive agents such as calcineurin inhibitors are administered after transplantation. Unfortunately, it is so far not possible to selectively inhibit the pathogenic immune cells and concomitantly leave the beneficial immune responses intact. As a consequence, all immune reactions, including the ones to infections or tumors, are prevented. This introduces the potential need for additional cell transfer to replace the immunosuppressed beneficial responses. However, since the immunosuppression has to be maintained to prevent transplant rejection of GvHD, the transferred cells then need a resistance to the immunosuppressive agents such as calcineurin inhibitors because otherwise they would get inhibited by CNI.

The inventors showed that the miR-17˜92 cluster and signalling via CD28 influences the sensitivity to Cyclosporin A. Thus, immune cells in which the expression of the miR-17˜92 cluster is increased or in which at least one miR-17˜92 target gene, e.g. Rcan3 (an endogenous calcineurin inhibitor) is deleted can be used in adoptive cell transfer therapy in a subject in combination with a calcineurin inhibitor.

By “immunosuppressive agent” it is intended an agent that suppresses immune function by one of several mechanisms of action. In other words, an immunosuppressive agent is a role played by a compound which is exhibited by a capability to diminish the extent and/or voracity of an immune response. Calcineurin inhibitor according to the invention is an immunosuppressive agent which functions by blocking directly or indirectly the calcineurin/NFAT pathway. The calcineurin inhibitor can be Cyclosporin A or FK506, also named Tacrolimus. According to the invention, the calcineurin inhibitor can also be CTLA4-Ig. Calcineurin (PP2B) is an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus, dimerize with other transcription factors and initiate the transcription of key target genes such as IL-2. Certain genes are activated by NFAT binding while others are inhibited. FK506 in complex with FKBP12, or cyclosporine A (CsA) in complex with CyPA block NFAT access to calcineurin's active site, preventing its dephosphorylation and thereby inhibiting T-cell activation (Brewin, Mancao et al. 2009). CTLA-4-Ig blocks NFAT activity through inhibition of T-cell CD28 costimulation (Diehn M. et al. Proc Natl Acad Sci USA. 2002, 99(18):11796-11801; Wang C J. Et al. Proc Natl Acad Sci USA. 2015; 112(2):524-9).

According to the present invention, the calcineurin inhibitor resistance is conferred to the immune cells by increasing the regulatory activity of the miR-17˜92 cluster or paralogs thereof. By “regulatory activity of miRNA”, it is intended the induction of target gene repression through binding and degradation of the target transcripts or by preventing mRNA from being translated.

In particular, according to the invention at least one miRNA of the miR7-92 cluster or paralogs thereof induce increased gene repression of at least one target gene through binding and subsequent degradation of the target transcript or by preventing mRNA from being translated. The miR7-92 target genes include but are not limited to the list of genes in Table 1.

TABLE 1
List of potential miR-17~92 cluster target genes.

| ENTREZ ID | SYMBOL | GENE NAME |
|---|---|---|
| 2247036 | March2 | Membrane - associated ring finger (C3HC4) 2 |
| 231570 | A830010M20Rik | RIKEN cDNA A830010M20 gene |
| 11308 | Abi1 | Abl-interactor 1 |
| 66885 | Acadsb | acyl-Coenzyme A dehydrogenase, short/branched chain |
| 68465 | Adipor2 | adiponectin receptor 2 |
| 11566 | Adss | adenylosuccinate synthetase, non muscle |
| 236511 | Ago1 | argonaute RISC catalytic subunit 1 |
| 14339 | Aktip | thymoma viral proto-oncogene 1 interacting protein |
| 70797 | Ankib1 | ankyrin repeat and IBR domain containing 1 |
| 433667 | Ankrd13c | ankyrin repeat domain 13c |
| 228359 | Arhgap1 | Rho GTPase activating protein 1 |
| 234094 | Arhgef10 | Rho guanine nucleotide exchange factor (GEF) 10 |
| 71704 | Arhgef3 | Rho guanine nucleotide exchange factor (GEF) 3 |
| 100504663 | Atg14 | autophagy related 14 |
| 109168 | At13 | atlastin GTPase 3 |
| 20238 | Atxn1 | ataxin 1 |
| 192197 | Bcas3 | breast carcinoma amplified sequence 3 |
| 52592 | Brms11 | breast cancer metastasis-suppressor 1-like |
| 100383 | Bsdc1 | BSD domain containing 1 |
| 70533 | Btf314 | basic transcription factor 3-like 4 |
| 77644 | C330007P06Rik | RIKEN cDNA C330007P06 gene |
| 232196 | C87436 | expressed sequence C87436 |
| 12380 | Cast | calpastatin |
| 52609 | Cbx7 | chromobox 7 |
| 216527 | Ccm2 | Cerebral cavernous malformation 2 |
| 212139 | Cc2d1a | coiled-coil and C2 domain containing 1A |
| 108686 | Ccdc88a | coiled coil domain containing 88A |
| 216527 | Ccm2 | cerebral cavernous malformation 2 |
| 217946 | Cdca71 | cell division cycle associated 7 like |
| 78334 | Cdk19 | cyclin-dependent kinase 19 |
| 14007 | Celf2 | CUGBP, Elav-like family member 2 |
| 74360 | Cep57 | centrosomal protein 57 |
| 12753 | Clock | circadian locomotor output cycles kaput |
| 104625 | Cnot6 | CCR4-NOT transcription complex, subunit 6 |
| 231464 | Cnot6l | CCR4-NOT transcription complex, subunit 6-like |
| 232430 | Crebl2 | cAMP responsive element binding protein-like 2 |
| 74114 | Crot | carnitine O-octanoyltransferase |
| 214897 | Csnk1g1 | casein kinase 1, gamma 1 |
| 74256 | Cyld | CYLD lysine 63 deubiquitinase |
| 225995 | D030056L22Rik | RIKEN cDNA D030056L22 gene |
| 98193 | Dcaf8 | DDB1 and CUL4 associated factor 8 |
| 67487 | Dhx40 | DEAH (Asp-Glu-Ala-His) box polypeptide 40 |
| 57431 | Dnajc4 | DnaJ heat shock protein family (Hsp40) member C4 |
| 75221 | Dpp3 | Dipeptidylpeptidase 3 |
| 13555 | E2f1 | E2F transcription factor 1 |
| 76740 | Efr3a | EFR3 homolog A |
| 68801 | Elov15 | ELOVL family member 5, elongation of long chain fatty |
| | | acids (yeast) |
| 83965 | Enpp5 | ectonucleotide pyrophosphatase/phosphodiesterase 5 |
| 67276 | Eri1 | exoribonuclease 1 |
| 52635 | Esyt2 | Extended synaptotagmin-like protein 2 |
| 104156 | Etv5 | ets variant 5 |
| 14055 | Ezh1 | enhancer of zeste 1 polycomb repressive complex 2 subunit |
| 213056 | Fam126b | family with sequence similarity 126, member B |
| 70186 | Fam162a | family with sequence similarity 162, member A |
| 67017 | Fam210b | family with sequence similarity 210, member B |
| 27999 | Fam3c | family with sequence similarity 3, member C |
| 67894 | Fam45a | family with sequence similarity 45, member A |
| 208836 | Fanci | Fanconi anemia, complementation group I |
| 50789 | Fbx13 | F-box and leucine-rich repeat protein 3 |
| 242960 | Fbx15 | F-box and leucine-rich repeat protein 5 |
| 319701 | Fbxo48 | F-box protein 48 |
| 218503 | Fcho2 | FCH domain only 2 |
| 216742 | Fnip1 | folliculin interacting protein 1 |
| 215751 | Ginm1 | glycoprotein integral membrane 1 |
| 83924 | Gpr137b | G protein-coupled receptor 137B |
| 73389 | Hbp1 | high mobility group box transcription factor 1 |
| 80517 | Herpud2 | HERPUD family member 2 |
| 15374 | Hn1 | hematological and neurological expressed sequence 1 |
| 320191 | Hook3 | hook microtubule tethering protein 3 |
| 231070 | Insig1 | insulin induced gene 1 |
| 69046 | Isca1 | iron-sulfur cluster assembly 1 |
| 16451 | Jak1 | Janus kinase 1 |
| 231986 | Jazf1 | JAZF zinc finger 1 |
| 71819 | Kif23 | kinesin family member 23 |
| 17113 | M6pr | mannose-6-phosphate receptor, cation dependent |
| 338372 | Map3k9 | mitogen-activated protein kinase kinase kinase 9 |
| 67121 | Mastl | microtubule associated serine/threonine kinase-like |
| 108645 | Mat2b | methionine adenosyltransferase II, beta |
| 98682 | Mfsd6 | major facilitator superfamily domain containing 6 |
| 338366 | Mia3 | melanoma inhibitory activity 3 |
| 216001 | Micu1 | mitochondrial calcium uptake 1 |
| 54484 | Mkrn1 | makorin, ring finger protein, 1 |
| 76763 | Mospd 2 | Motile sperm domain containing 2 |
| 67468 | Mmd | monocyte to macrophage differentiation-associated |
| 17764 | Mtf1 | metal response element binding transcription factor 1 |
| 56174 | Nagk | N-acetylglucosamine kinase |
| 240055 | Neurl1b | neuralized E3 ubiquitin protein ligase 1B |
| 18020 | Nfatc2ip | nuclear factor of activated T cells, cytoplasmic, calcineurin |
| | | dependent 2 interacting protein |
| 192292 | Nrbp1 | nuclear receptor binding protein 1 |
| 79196 | Osbp15 | oxysterol binding protein-like 5 |
| 170719 | Oxr1 | oxidation resistance 1 |
| 319263 | Pcmtd1 | protein-L-isoaspartate (D-aspartate) O-methyltransferase |
| | | domain containing 1 |
| 236899 | Pcyt1b | phosphate cytidylyltransferase 1, choline, beta isoform |
| 236899 | Pcyt1b | phosphate cytidylyltransferase 1, choline, beta isoform |
| 58205 | Pdcd1lg2 | programmed cell death 1 ligand 2 |
| 242202 | Pde5a | phosphodiesterase 5A, cGMP-specific |
| 244650 | Phlpp2 | PH domain and leucine rich repeat protein phosphatase 2 |
| 74769 | Pik3cb | phosphatidylinositol 3-kinase, catalytic, beta polypeptide |
| 233765 | Plekha7 | pleckstrin homology domain containing, family A member |
| | | 7 |
| 353047 | Plekhm1 | pleckstrin homology domain containing, family M (with |
| | | RUN domain) member 1 |
| 102595 | Plekho2 | pleckstrin homology domain containing, family O member |
| | | 2 |
| 108767 | Pnrc1 | proline-rich nuclear receptor coactivator 1 |
| 330260 | Pon2 | paraoxonase 2 |
| 73825 | Ppp1r21 | protein phosphatase 1, regulatory subunit 21 |
| 67229 | Prpf18 | pre-mRNA processing factor 18 |
| 218699 | Pxk | PX domain containing serine/threonine kinase |
| 19338 | Rab33b | RAB33B, member RAS oncogene family |
| 19344 | Rab5b | RAB5B, member RAS oncogene family |
| 19357 | Rad21 | RAD21 cohesin complex component |
| 76089 | Rapgef2 | Rap guanine nucleotide exchange factor (GEF) 2 |
| 19418 | Rasgrf2 | RAS protein-specific guanine nucleotide-releasing factor 2 |
| 53902 | Rcan3 | regulator of calcineurin 3 |
| 19731 | Rgl1 | ral guanine nucleotide dissociation stimulator,-like 1 |
| 29864 | Rnf11 | ring finger protein 11 |
| 70510 | Rnf167 | ring finger protein 167 |
| 73469 | Rnf38 | ring finger protein 38 |
| 56613 | Rps6ka4 | ribosomal protein S6 kinase, polypeptide 4 |
| 54650 | Sfmbt1 | Scm-like with four mbt domains 1 |
| 27059 | Sh3d19 | SH3 domain protein D19 |
| 20499 | Slc12a7 | solute carrier family 12, member 7 |
| 98396 | Slc41a1 | solute carrier family 41, member 1 |
| 20544 | Slc9a1 | solute carrier family 9 (sodium/hydrogen exchanger), |
| | | member 1 |
| 52864 | Slx4 | SLX4 structure-specific endonuclease subunit homolog (S. |
| | | cerevisiae) |
| 75627 | Snapc1 | small nuclear RNA activating complex, polypeptide 1 |
| 266781 | Snx17 | sorting nexin 17 |
| 223918 | Spryd3 | SPRY domain containing 3 |
| 170459 | Stard4 | StAR-related lipid transfer (START) domain containing 4 |
| 20744 | Strbp | spermatid perinuclear RNA binding protein |
| 58244 | Stx6 | syntaxin 6 |
| 229521 | Syt11 | synaptotagmin XI |
| 21813 | Tgfbr2 | transforming growth factor, beta receptor II |
| 21815 | Tgif1 | TGFB-induced factor homeobox 1 |
| 21844 | Tiam1 | T cell lymphoma invasion and metastasis 1 |
| 330401 | Tmcc1 | transmembrane and coiled coil domains 1 |
| 71929 | Tmem123 | transmembrane protein 123 |
| 69470 | Tmem127 | transmembrane protein 127 |
| 77975 | Tmem50b | transmembrane protein 50B |
| 100201 | Tmem64 | transmembrane protein 64 |
| 74868 | Tmem65 | transmembrane protein 65 |
| 228140 | Tnks1bp1 | tankyrase 1 binding protein 1 |
| 30934 | Tor1b | torsin family 1, member B |
| 70827 | Trak2 | trafficking protein, kinesin binding 2 |
| 109161 | Ube2q2 | ubiquitin-conjugating enzyme E2Q family member 2 |
| 216558 | Ugp2 | UDP-glucose pyrophosphorylase 2 |
| 22388 | Wdr1 | WD repeat domain 1 |
| 226757 | Wdr26 | WD repeat domain 26 |
| 11798 | Xiap | X-linked inhibitor of apoptosis |
| 75580 | Zbtb4 | zinc finger and BTB domain containing 4 |
| 170740 | Zfp287 | zinc finger protein 287 |
| 238673 | Zfp367 | zinc finger protein 367 |
| 71063 | Zfp597 | zinc finger protein 597 |
| 68520 | Zfyve21 | zinc finger, FYVE domain containing 21 |
| 66505 | Zmynd11 | zinc finger, MYND domain containing 11 |

In a particular embodiment, at least one miRNA of the miR17˜92 cluster or paralogs thereof induce an increase of gene repression of regulators of calcineurin genes, such as RCAN1, RCAN2 and RCAN3, preferably RCAN3 gene.

The increase of the regulatory activity of the miR-17˜92 cluster may be determined by measuring the expression level of target genes of miR-17˜92 cluster or paralogs thereof. In a particular embodiment, the target gene is selected in the list of genes in Table 1. In a more particular embodiment, the target gene is selected from the group consisting of RCAN3 gene, Cast gene, Cyld gene and Zbtb4 gene, more preferably RCAN3 gene. The regulatory activity of the miR-17˜92 cluster or paralogs thereof is increased in engineered immune cells when the expression level of the target gene, preferably selected from the group consisting of RCAN3 gene, Cast gene, Cyld gene and Zbtb4 gene, more preferably RCAN3 gene is at least 1.5-fold lower, or 2, 3, 4, 5-fold lower than in non-engineered immune cells.

The expression level of mRNA may be determined by any suitable methods known by skilled persons. Usually, these methods comprise measuring the quantity of mRNA. Methods for determining the quantity of mRNA are well known in the art. For example the nucleic acid contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred.

The level of the target genes protein may also be determined by any suitable methods known by skilled persons. Usually, these methods comprise contacting a cell sample, preferably a cell lysate, with a binding partner capable of selectively interacting with the target gene protein present in the sample. The binding partner is generally a polyclonal or monoclonal antibodies, preferably monoclonal. The quantity of the protein may be measured, for example, by semi-quantitative Western blots, enzyme-labelled and mediated immunoassays, such as ELISAs, biotin/avidin type assays, radioimmunoassay, immunoelectrophoresis or immunoprecipitation or by protein or antibody arrays. The reactions generally include revealing labels such as fluorescent, chemiluminescent, radioactive, enzymatic labels or dye molecules, or other methods for detecting the formation of a complex between the antigen and the antibody or antibodies reacted therewith. In a preferred embodiment, cell lysate is digested with specific enzymes, for example trypsin, to receive fractionated proteins. The samples are then mixed with spiking peptides. The spiking peptides are defined as short peptides derived from the protein of interest, with isotope labelling. Subsequent measuring with mass spectrometry then detects a mass shift and enables to quantify the abundance of the protein of interest.

In another particular embodiment, the increase of the regulatory activity of the miR-17˜92 cluster may be determined by measuring the expression level of at least one of the miRNAs of the miR-17˜92 cluster and paralogs thereof. The regulatory activity of the miR-17˜92 cluster is increased in engineered immune cells when the expression level of at least one miRNAs of miR-17˜92 cluster and paralogs thereof is at least 1.5-fold higher, or 2, 3, 4, 5-fold higher than in non-engineered immune cells. The expression level of miRNA can be determined by any suitable methods known by skilled persons as described above.

The regulatory activity or amount of miR-17˜92 cluster and paralogs can be increased by agents which include, but are not limited to, chemicals, antibiotic, compounds known to modify gene expression, modified or unmodified polynucleotides (including oligonucleotides), polypeptides, peptides, small RNA molecules and miRNAs.

In a particular embodiment, the regulatory activity of miR-17˜92 cluster and paralogs thereof is increased by engineered immune cell to overexpress at least one miRNA of the miR-17˜92 cluster and paralogs thereof.

As used herein, the term “overexpress” or “overexpression” refers to an expression level which is, after normalization, at least 1.5-fold higher, or 2, 3, 4, 5-fold higher, than the expression level in non-modified immune cells. Expression levels may be normalized by using expression levels of mRNA which are known to have stable expression such as ribosomal 18S, GAPDH (glyceraldehyde 3-phosphate dehydrogenase) or β-actin.

Overexpression of miR17˜92 cluster in immune cells may be obtained by any methods known by the skilled person such as by increasing the transcription of MIR17HG gene or paralogs thereof for example by using CRISPR activation system with a dCas9 without endonuclease activity and added transcriptional activators on dCas9 or the guide RNA. Overexpression of miR17˜92 cluster in immune cells may also be obtained by introducing a nucleic acid construct or vector comprising a nucleic acid sequence encoding at least one miRNA of the miR17˜92 cluster and paralogs thereof, and/or decreasing the degradation of said miRNA.

The term “miRNA” or “microRNA” or “miR” encompasses single-stranded and double-stranded miRNAs. Preferably, the miRNAs are of double-stranded form. The miRNAs are partially complementary to their target mRNAs and have a size between 10 and 25 nucleotides, preferably between 20 and 25 nucleotides.

The miRNAs used according to the invention can be of the single-stranded or double-stranded form or a mixture of the two. They can contain modified nucleotides or chemical modifications enabling them, for example, to increase their resistance to nucleases and thus increase their lifetime in the cell. They can notably comprise at least one modified or non-natural nucleotide such as, for example, a nucleotide having a modified base, such as inosine, methyl-5-deoxycytidine, dimethylamino-5-deoxyuridine, deoxyuridine, diamino-2,6-purine, bromo-5-deoxyuridine or any other modified base permitting hybridization. The interfering RNAs used according to the invention can also be modified at the internucleotide bond, for example phosphorothioates, H-phosphonates or alkyl phosphonates, or in the backbone, for example alpha-oligonucleotides, 2′-O-alkyl ribose or PNAs (peptide nucleic acids) (M. Egholm et al., 1992).

The interfering RNAs can be natural RNAs, synthetic RNAs or those produced by recombination techniques. These miRNAs can be prepared by any methods known to a person skilled in the art, such as, for example, chemical synthesis, screening of databases, transcription in vivo or recombinant DNA or amplification techniques.

The miR-17˜92 cluster is a polycistronic transcript (SEQ ID NO: 1; GenBank: AB176708.1) encoded by the MIR17HG gene, also named C13orf25. The miR-17˜92 cluster comprises a group of six miRNAs: miR-17, miR-18a, miR-19a, miR-20, miR-19b and miR-92.

Two paralogs of the miR-17˜92 cluster have been identified, miR-106a-363 and miR-106b-25 clusters. The miR-106a-363 cluster is located on chromosome X and encodes six miRNAs: miR-106a, miR-18b, miR-19b-2, miR-20b, miR-92a-2, and miR-363. The cluster miR106b-25 encodes three miRNAs: miR-106b, miR-93, and miR-25.

According to the present invention, by “miR-17˜92 cluster” it is intended the miR-17˜92 cluster as well as paralogs thereof such as miR-106a-363 and miR-106b-25 clusters. As used herein, the term “paralogs” indicates separate occurrences of a gene (or other coding sequence) in one species. The separate occurrences have similar, albeit nonidentical, sequences, the degree of sequence similarity depending, in part, upon the evolutionary distance from the gene duplication event giving rise to the separate occurrences. The term “paralogs” hence refers to naturally occurring variants.

The miRNAs used according to the invention can be administered in the form of precursors. The miRNAs can notably be administered in the form of pre-miRNAs or of pri-miRNAs. The pri-miRNAs are precursors of miRNA which are cleaved in the nucleus of the cells to form pre-miRNAs. A pri-miRNA can comprise one or more pre-miRNAs. The pre-miRNAs are also precursors of the miRNAs. They comprise between 60 and 80 nucleotides and are folded into an imperfect stem-and-loop structure. These pre-miRNAs are cleaved in the cytoplasm to form double-stranded miRNAs and then single-stranded miRNAs capable of interacting with a protein of the Argonaute family to form the RISC complex, owing to which either the target mRNA is degraded, or the translation of this mRNA is repressed. The processing of a pre-miRNA into mature miRNAs results in two 19-23 nt long miRNAs named miR-XXX-5p and miR-XXX-3p; the mature miR-XXX-5p miRNA originates from 5′-end and miR-XXX-3p originates from 3-end of the pre-miRNA. The term “miRNA” includes reference to Pri-miRNAs, pre-mRNAs, 5p-miRNa and 13p-miRNA.

The precursors miRNAs of the miR-17˜92 cluster and paralogs tat can be used in the present disclosure are listed in the Table 2.

TABLE 2
The precursors miRNAs of the miR-17-92 cluster and paralogs

| Precursor miRNAs | Nucleotide sequence | SEQ ID | reference |
|---|---|---|---|
| Pre-miR-17 | gtcagaataa tgtcaaagtg cttacagtgc aggtagtgat | 2 | NR_029487.1 |
| | atgtgcatct actgcagtga aggcacttgt agcattatgg | | |
| | tgac | | |
| Pre-miR-18a | tgttctaagg tgcatctagt gcagatagtg aagtagatta | 3 | NR_029488.1 |
| | gcatctactg ccctaagtgc tccttctggc a | | |
| Pre-miR-19a | gcagtcctct gttagttttg catagttgca ctacaagaag | 4 | NR_029489.1 |
| | aatgtagttg tgcaaatcta tgcaaaactg atggtggcct gc | | |
| Pre-miR-20a | gtagcactaa agtgcttata gtgcaggtag tgtttagtta | 5 | NR_029492.1 |
| | tctactgcat tatgagcact taaagtactg c | | |
| Pre-miR-19b-1 | cactgttcta tggttagttt tgcaggtttg catccagctg | 6 | NR_029490.1 |
| | tgtgatattc tgctgtgcaa atccatgcaa aactgactgt | | |
| | ggtagtg | | |
| Pre-miR92a-1 | ctttctacac aggttgggat cggttgcaat gctgtgatc | 7 | NR_029508.1 |
| | tgtatggtat tgcacttgtc ccggcctgtt gagtttgg | | |
| Pre-miR-106a | ccttggccat gtaaaagtgc ttacagtgca ggtagctttt | 8 | GenBank: |
| | tgagatctac tgcaatgtaa gcacttctta cattaccatg g | | LM608196.1 |
| Pre-miR-18b | tgtgttaagg tgcatctagt gcagttagtg aagcagctta | 9 | NR_029949.1 |
| | gaatctactg ccctaaatgc cccttctggc a | | |
| pre-miR-19b-2 | acattgctac ttacaattag ttttgcaggt ttgcatttca | 10 | GenBank: |
| | gcgtatatat gtatatgtgg ctgtgcaaat ccatgcaaaa | | LM608164.1 |
| | ctgattgtga taatgt | | |
| Pre-miR-20b | agtaccaaag tgctcatagt gcaggtagtt ttggcatgac | 11 | NR_029950.1 |
| | tctactgtag tatgggcact tccagtact | | |
| Pre-miR-92a-2 | tcatccctgg gtggggattt gttgcattac ttgtgttcta | 12 | NR_029509.1 |
| | tataaagtat tgcacttgtc ccggcctgtg gaaga | | |
| Pre-miR-363 | tgttgtcggg tggatcacga tgcaattttg atgagtatca | 13 | NR_029950.1 |
| | taggagaaaa attgcacggt atccatctgt aaacc | | |
| Pre-miR-106b | cctgccgggg ctaaagtgct gacagtgcag atagtggtcc | 14 | GenBank: |
| | tctccgtgct accgcactgt gggtacttgc tgctccagca | | LM608661.1 |
| | gg | | |
| Pre-miR-93 | ctgggggctc caaagtgctg ttcgtgcagg tagtgtgatt | 15 | NR_029510.1 |
| | acccaaccta ctgctgagct agcacttccc gagcccccgg | | |
| Pre-miR-25 | ggccagtgtt gagaggcgga gacttgggca attgctggac | 16 | NR_029498.1 |
| | gctgccctgg gcattgcact tgtctcggtc tgacagtgcc | | |
| | ggcc | | |

Thus, the precursor miRNA of the miR-192 is selected from the group consisting of: pre-miR-17 (SEQ ID NO: 2; NCBI reference: NR_029487.1), pre-miR-18a (SEQ ID NO: 3; NCBI reference: NR_029488.1), pre-miR-19a (SEQ ID NO: 4; NCBI reference: NR_029489.1), pre-miR-20a (SEQ ID NO: 5; NCBI reference: NR_029492.1), pre-miR-19b-1 (SEQ ID NO: 6; NCBI reference: NR_029490.1) and pre-miR-92a-1 (SEQ ID NO: 7; NCBI reference: NR_029508.1).

The precursor miRNAs of miR17˜92 clusters paralog is selected from the group consisting of: pre-miR-106a (SEQ ID NO: 8, GenBank: LM608196.1), pre-miR-18b (SEQ ID NO: 9, NCBI Reference Sequence: NR_029949.1), pre-miR-19b-2 (SEQ ID NO: 10, GenBank: LM608164.1), pre-miR-20b (SEQ ID NO: 11, NCBI Reference Sequence: NR_029950.1), pre-miR-92a-2 (SEQ ID NO: 12, NCBI Reference Sequence: NR_029509.1), pre-miR-363 (SEQ ID NO: 13, NCBI Reference Sequence: NR_029852.1), pre-miR-106b (SEQ ID NO: 14, GenBank: LM608661.1), pre-miR-93 (SEQ ID NO: 15, NCBI Reference Sequence: NR_029510.1) and pre-miR-25 (SEQ ID NO: 16, NCBI Reference Sequence: NR_029498.1).

The miR-17˜92 cluster and paralogs thereof comprises at least one miRNA selected from the list of Table 3 below.

TABLE 3
miRNAs of the miR-17-92 cluster and paralogs.

| | | | | Nucleotide | |
|---|---|---|---|---|---|
| miRNA | Nucleotide sequence | SEQ ID | miRNA | sequence | SEQ ID |
| miR-17-5p | caaagtgctt acagtg- | 17 | miR-17-3p | actgcagtga ag- | 18 |
| | cagg tag | | | gcacttgt ag | |
| miR-18a-5p | taaggtgcat ctagtgcaga | 19 | miR18a-3p | actgccctaa | 20 |
| | tag | | | gtgctccttc tgg | |
| miR-19a-5p | agttttgcat agttgcacta | 21 | miR-19a-3p | tgtgcaaatc | 22 |
| | ca | | | tatgcaaaac tga | |
| miR-20a-5p | taaagtgctt atagtgcagg | 23 | miR-20a-3p | actgcattat gagcac- | 24 |
| | tag | | | ttaa ag | |
| miR-19b-1-5p | agttttgcag gtttgcatcc | 25 | miR-19b-1-3p | tgtgcaaatc | 26 |
| | agc | | | catgcaaaac tga | |
| miR-92a-1-5p | aggttgggat cggttgcaat | 27 | miR-92a-1-3p | tattgcactt | 28 |
| | gct | | | gtcccggcct gt | |
| miR-106a-5p | aaaagtgctt acagtg- | 29 | miR-106a-3p | ctgcaatgta agcac- | 30 |
| | cagg tag | | | ttctt ac | |
| miR-18b-5p | taaggtgcat ctagtgcagt | 31 | miR-18b-3p | tgccctaaat | 32 |
| | tag | | | gccccttctg gc | |
| miR-19b-2-5p | agttttgcag gtttgcattt | 33 | miR-19b-2-3p | tgtgcaaatc | 34 |
| | ca | | | catgcaaaac tga | |
| miR-20b-5p | caaagtgctc atagtg- | 35 | miR-20b-3p | actgtagtat gggcac- | 36 |
| | cagg tag | | | ttcc ag | |
| miR-92a-2-5p | gggtggggat ttgttgcatt | 37 | miR-92a-2-3p | tattgcactt | 38 |
| | ac | | | gtcccggcct gt | |
| miR-363-5p | cgggtggatc ac- | 39 | miR-363-3p | aattgcacgg | 40 |
| | gatgcaat tt | | | tatccatctg ta | |
| miR106b-5p | taaagtgctg acagtg- | 41 | miR106b-3p | ccgcactgtg | 42 |
| | caga t | | | ggtacttgct gc | |
| miR-93-5p | caaagtgctg ttcgtgcagg | 43 | miR-93-3p | actgctgagc tag- | 44 |
| | tag | | | cacttcc cg | |
| miR-25-5p | aggcggagac ttggg- | 45 | miR-25-3p | cattgcactt | 46 |
| | caatt g | | | gtctcggtct ga | |

The miR-17˜92 cluster and paralogs thereof comprises at least one miRNA selected from the group consisting of: miR-17-5p (SEQ ID NO: 17), miR-17-3p (SEQ ID NO: 18), miR-18a-5p (SEQ ID NO: 19), miR18a-3p (SEQ ID NO: 20), miR-19a-5p (SEQ ID NO: 21), miR-19a-3p (SEQ ID NO: 22) miR-20a-5p (SEQ ID NO: 23), miR-20a-3p (SEQ ID NO: 24), miR-19b-1-5p (SEQ ID NO: 25) and miR-19b-1-3p (SEQ ID NO: 26) and miR-92a-1-5p (SEQ ID NO: 27), miR-92a-1-3p (SEQ ID NO: 28), miR-106a-5p (SEQ ID NO: 29), miR-106a-3p (SEQ ID NO: 30), miR-18b-5p (SEQ ID NO: 31), miR-18b-3p (SEQ ID NO: 32), miR-19b-2-5p (SEQ ID NO: 33), miR-19b-2-3p (SEQ ID NO: 34), miR-20b-5p (SEQ ID NO: 35), miR-20b-3p (SEQ ID NO: 36), miR-92a-2-5p (SEQ ID NO: 37), miR-92a-2-3p (SEQ ID NO: 38), miR-363-5p (SEQ ID NO: 39), miR-363-3p (SEQ ID NO: 40), miR-106b-5p (SEQ ID NO: 41), miR-106b-3p (SEQ ID NO: 42), miR-93-5p (SEQ ID NO: 43), miR-93-3p (SEQ ID NO: 44), miR-25-5p (SEQ ID NO: 45) and miR-25-3p (SEQ ID NO: 46).

In a preferred embodiment, the immune cell is genetically engineered by introducing into the immune cell a nucleic acid construct comprising at least one miRNA sequence, preferably 2, 3, 4, 5, 6, 7, 8, 9, 10, 12, 15, 20, 25, 30 selected from the group consisting of: SEQ ID NO: 17 to 46, preferably SEQ ID NO: 17, 18, 21, 22, 25 and 26. In a particular embodiment, said nucleic acid construct comprises at least one pre-miRNA sequence, more preferably 2, 3, 4, 5, 6, 7, 8, 9, 10, 11, 12, 13, 14, 15, 16 selected from the group consisting of: SEQ ID NO: 1 to 16, preferably SEQ ID NO: 2, 4 and 6.

The terms “nucleic acid sequence” and “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the invention.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e. a nucleic acid molecule which is used to deliver exogenously created DNA into a host cell.

Generally, the nucleic acid construct comprises a coding sequence and regulatory sequences preceding (5′ non-coding sequences) and following (3′ non-coding sequences) the coding sequence that are required for expression of the selected gene product. Thus, a nucleic acid construct typically comprises a promoter sequence, a coding sequence and a 3′ untranslated region that usually contains a polyadenylation site and/or transcription terminator. The nucleic acid construct may also comprise additional regulatory elements such as, for example, enhancer sequences, a polylinker sequence facilitating the insertion of a DNA fragment within a vector and/or splicing signal sequences.

In one embodiment, the nucleic acid construct comprises a promoter. Said promoter initiates transgene expression upon introduction into a host cell. As used herein, the term “promoter” refers to a regulatory element that directs the transcription of a nucleic acid to which it is operably linked. A promoter can regulate both rate and efficiency of transcription of an operably linked nucleic acid. A promoter may also be operably linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which is operably linked, on the same strand and upstream of the DNA sequence (towards the 5′ region of the sense strand). A promoter can be about 100-1000 base pairs long. Positions in a promoter are designated relative to the transcriptional start site for a particular gene (i.e., positions upstream are negative numbers counting back from −1, for example −100 is a position 100 base pairs upstream).

As used herein, the term “operably linked” refers to a linkage of polynucleotide (or polypeptide) elements in a functional relationship. A nucleic acid is “operably linked” when it is placed into a functional relationship with another nucleic acid sequence. For instance, a promoter or transcription regulatory sequence is operably linked to a coding sequence if it affects the transcription of the coding sequence. Operably linked means that the DNA sequences being linked are typically contiguous; where it is necessary to join two protein encoding regions, they are contiguous and in reading frame.

In another particular embodiment said nucleic acid construct is a vector. Examples of appropriate vectors include, but are not limited to, recombinant integrating or non-integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example.

Nucleic acid molecule or nucleic acid construct, expression cassette or vector according to the invention may be transferred into immune cells using any known technique including, but being not limited to calcium phosphate transfection, DEAE-Dextran transfection, electroporation, microinjection, biolistic, viral infection or liposome-mediated transfection. In a preferred embodiment, the RNA, preferably miRNA can be produced in vitro by e.g. in vitro transcription. The RNA may then be introduced into the immune cells by electroporation (as described for example in Almasbak et al., Cytotherapy 2011, 13:629-640; Rabinovich et al., Hum Gene Ther, 2009, 2:51-60; and Beatty et al., Cancer Immunol Res 2014, 2, 1:12-120). Alternatively, RNA may be introduced by other means such as by liposomes or cationic molecules etc. In another embodiment, nucleic acid construct or vector introduced into a cell may be expressed episomally, or may be integrated into the genome of the cell.

In another particular embodiment, the regulatory activity of miR-17˜92 cluster and paralogs thereof is increased by engineering immune cell to decrease at least one miR-17˜92 target gene activity. Said target genes include but are not limited to the genes listed in the Table 1 above. In particular, said target gene is selected from the group consisting of RCAN1, RCAN2, RCAN3 gene, Cast gene, Cyld gene and Zbtb4 gene, preferably RCAN3 gene.

In a particular embodiment, said immune cell is engineered to inactivate or repress the expression of at least one miR-17˜92 cluster target gene. The inactivation of said target gene is preferably performed by a genome modification, more particularly through the introduction in the immune cell of a specific rare-cutting endonuclease able to target a genetic locus directly or indirectly involved in the production miR-17˜92 target gene. Different types of rare-cutting endonucleases can be used, such as Meganucleases, TAL-nucleases, zing-finger nucleases (ZFN), or RNA/DNA guided endonucleases like Cas9/CRISPR or Argonaute.

By inactivating a target gene it is intended that the gene of interest is not or less expressed in a functional protein form. In particular embodiment, the genetic modification of the method relies on the introduction, in provided cells to engineer, of one rare-cutting endonuclease such that said rare-cutting endonuclease specifically catalyzes cleavage in one targeted gene thereby inactivating said targeted gene.

The term “rare-cutting endonuclease” refers to a wild type or variant enzyme capable of catalyzing the hydrolysis (cleavage) of bonds between nucleic acids within a DNA or RNA molecule, preferably a DNA molecule. In a particular embodiment, said rare-cutting endonuclease according to the present invention is a RNA-guided endonuclease such as the Cas9/CRISPR complex. RNA guided endonucleases is a genome engineering tool where an endonuclease associates with a RNA molecule. In this system, the RNA molecule nucleotide sequence determines the target specificity and activates the endonuclease (Gasiunas, Barrangou et al. 2012; Jinek, Chylinski et al. 2012; Cong, Ran et al. 2013; Mali, Yang et al. 2013).

In a preferred embodiment, said immune cell is engineered to inactivate the expression of at least one miR-17˜92 cluster target gene, preferably by introducing Cas9/CRISPR complex able to target at least one miR-17˜92 cluster target gene, more preferably by introducing into said immune cell a a Cas9 nuclease and a guide RNA, also referred here as single guide RNA. Said single guide RNA is preferably able to target at least one miR-17˜92 cluster target gene, more preferably selected from the group consisting of RCAN1, RCAN2, RCAN3 gene, Cast gene, Cyld gene and Zbtb4 gene, again more preferably Rcan3 gene. In a more preferred embodiment said single guide RNA is able to target Rcan3 gene.

The inactivation of said target gene can also be performed by the use of site-specific base editors, for example by introducing premature stop codon(s), deleting a start codon or altering RNA splicing. Base editing directly generates precise point mutations in DNA without creating DNA double strand breaks. In a particular embodiment, base editing is performed by using DNA base editors which comprise fusions between a catalytically impaired Cas nuclease and a base modification enzyme that operates on single-stranded DNA (for review, see Rees H. A. et al. Nat Rev Genet. 2018. 19(12):770-788.

In another embodiment, said immune cell is engineered to repress expression of miR-17˜92 cluster target genes. The repression mediated by the miR-17˜92 cluster may be based on anti-sense oligonucleotide constructs, small inhibitory RNAs (siRNAs), short hairpin RNA. Anti-sense oligonucleotides, including anti-sense RNA molecules and anti-sense DNA molecules, would act to directly block the translation of miR-17˜92 target gene mRNA by binding thereto and thus preventing protein translation or increasing mRNA degradation, thus decreasing the level of miR-17˜92 target gene, and thus activity, in a cell. For example, antisense oligonucleotides of at least about 15 bases and complementary to unique regions of the mRNA transcript sequence can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In another embodiment, small inhibitory RNAs (siRNAs) can also be used to decrease the miR-17˜92 cluster target gene expression level in the present invention. miR-17˜92 cluster target gene expression can be reduced by introducing into a cell a small double stranded RNA (dsRNA), or a vector or construct causing the production of a small double stranded RNA, such that miR-17˜92 cluster target gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In another embodiment, short hairpin RNA (shRNA) can also be used to decrease the miR-17˜92 cluster target gene expression level in the present invention. A short hairpin RNA (shRNA) is a sequence of RNA that makes a tight hairpin turn that can be used to silence target gene expression via RNA interference (RNAi). Expression of shRNA in cells is typically accomplished by delivery of plasmids or through viral or bacterial vectors. The promoter choice is essential to achieve robust shRNA expression. At first, polymerase III promoters such as U6 and HI were used; however, these promoters lack spatial and temporal control. As such, there has been a shift to using polymerase II promoters to regulate expression of shRNA.

In another embodiment, CRISPR interference can also be used to decrease the miR-17˜92 cluster target gene expression level in the present invention. CRISPR interference uses a catalytically dead Cas9 protein that lacks endonuclease activity but is coupled to transcriptional repressors to downregulate specific genes using RNA-guided repression. Targeting specificity is determined by complementary base-pairing of a single guide RNA (sgRNA) to the genomic loci. Preferably, the immune cell is engineered by introducing into the immune cell a nucleic acid construct encoding a Cas9 protein or other Cas that lacks endonuclease activity and a single guide RNA specific to miR17˜92 cluster target gene, preferably selected from the group consisting of RCAN1, RCAN2, RCAN3 gene, Cast gene, Cyld gene and Zbtb4 gene, preferably RCAN3 gene.

In a preferred embodiment, the immune cell is genetically engineered by introducing into the immune cell a nucleic acid construct as described above comprising said nuclease, anti-sense oligonucleotide constructs, small inhibitory RNAs (siRNAs) or short hairpin RNA. In a more preferred embodiment, said nucleic acid construct is a vector as described above.

In a more preferred embodiment, the immune cell is engineered by introducing into the immune cell a Cas9 nuclease and a guide RNA, also referred here as single guide RNA able to target at least one miR-17˜92 cluster target gene, preferably target gene selected from the group consisting of RCAN1, RCAN2, RCAN3 gene, Cast gene, Cyld gene and Zbtb4 gene, more preferably Rcan3 gene. Said Cas9 nuclease can be introduced into said cell by introducing a nucleic acid construct such as vector or mRNA encoding Cas9 nuclease or by directly introducing Cas9 protein into said cell. As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, the term “T cell” includes cells bearing a T cell receptor (TCR). T-cells according to the invention can be selected from the group consisting of inflammatory T-lymphocytes, cytotoxic T-lymphocytes, regulatory T-lymphocytes, tumour infiltrating lymphocytes or helper T-lymphocytes included both type 1 and 2 helper T cells and Th17 helper cells. In another embodiment, said cell can be derived from the group consisting of CD4+T-lymphocytes and CD8+T-lymphocytes or non-classical T cells such as MR1 restricted T cells, MAIT cells, MR1T cells, gamma delta T cells or innate-like T cells. Said immune cells may originate from a healthy donor or from a subject.

Immune cells can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person, such as FICOLL™ separation. In one embodiment, cells from the circulating blood of a subject are obtained by apheresis. In certain embodiments, T-cells are isolated from PBMCs. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes, for instance by using CD14 DYNABEADS®. In some embodiments, red blood cells may be lysed prior to the density gradient centrifugation.

In another embodiment, said cell can be derived from a healthy donor, from a subject diagnosed with cancer or an auto-immune disease or from a subject diagnosed with an infection.

Generally, the immune cells are activated and expanded to be utilized in the ACT therapy. The immune cells of the invention can be expanded in vivo or ex vivo. The immune cells, in particular T-cells can be activated and expanded generally using methods known in the art. Generally the T-cells are expanded by contact with a surface having attached thereto an agent that stimulates a CD3/TCR complex associated signal and a ligand that stimulates a co-stimulatory molecule on the surface of the T cells.

For use in adoptive cell transfer therapy, in a particular embodiment, the immune cell may be modified to display desired specificities and enhanced functionalities. In particular, the immune cell may be modified to be directed to a specific target. In a particular embodiment, said immune cell may express a recombinant antigen receptor on its cell surface. By “recombinant” is meant an antigen receptor which is not encoded by the cell in its native state, i.e. it is heterologous, non-endogenous. Expression of the recombinant antigen receptor can thus be seen to introduce a new antigen specificity to the immune cell, causing the cell to recognise and bind a previously unrecognised antigen. The antigen receptor may be isolated from any useful source.

In a particular embodiment, said recombinant antigen receptor is a recombinant T-Cell Receptor (TCR). TCR is present on the surface of T cells and is responsible for recognizing fragment of antigen as peptides bound to major histocompatibility complex (MHC) molecules. Most TCRs comprise an α- and a β-chain, both of which consist of a variable region and a constant region. The variable region is located at the N-terminus of the chain, and is wholly extracellular; the constant region is located at the C-terminus of the chain, and consists of an extracellular domain, a transmembrane domain and a short cytoplasmic domain. TCR chains are encoded and synthesised in an immature form, with an N-terminal signal (or leader) sequence. This sequence forms the N-terminus of the variable region of a α- or β-TCR chain when it is synthesised. Following synthesis of the TCR chain, the signal sequence is cleaved, and so is not present in a mature TCR located at the cell surface.

The variable region of a α- or β-chain comprises three hypervariable, complementarity determining regions (CDRs). These CDRs determine the specificity of the TCR, with CDR3 (that is, the third CDR from the N-terminus) being the most important CDR in determining TCR specificity. TCRs recognise specific MHC-antigen complexes. Upon binding of a TCR to its cognate MHC-antigen complex, the T-cell is stimulated to proliferate and its effector functions are activated. Thus a T-cell can be easily redirected by modification to express a recombinant TCR. Many TCRs of medical interest are known and have been used in clinical trials and therapy.

Another recombinant antigen receptor which is useful in the invention is a chimeric antigen receptor (CAR). CARs are fusion proteins comprising an antigen-binding domain, typically derived from an antibody, linked to the signalling domain of the TCR complex. CARs can be used to direct immune cells such T-cells or NK cells against a target antigen if a suitable antigen-binding domain is selected.

The antigen-binding domain of a CAR is typically based on a scFv (single chain variable fragment) derived from an antibody. In addition to an N-terminal, extracellular antibody-binding domain, CARs typically may comprise a hinge domain, which functions as a spacer to extend the antigen-binding domain away from the plasma membrane of the immune effector cell on which it is expressed, a transmembrane (TM) domain, an intracellular signalling domain (e.g. the signalling domain from the zeta chain of the CD3 molecule (CD3ζ) of the TCR complex, or an equivalent) and optionally one or more co-stimulatory domains which may assist in signalling or functionality of the cell expressing the CAR. Signalling domains from co-stimulatory molecules including CD28, OX-40 (CD134), and 4-1BB (CD137) can be added alone (second generation) or in combination (third generation) to enhance survival and increase proliferation of CAR modified T cells.

The skilled person is able to select an appropriate antigen receptor with which to redirect an immune cell to be used according to the invention. In a particular embodiment, the immune cell for use in the method of the invention is a redirected T-cell, e.g. a redirected CD8+ T-cell or a redirected CD4+ T-cell.

Methods by which immune cells can be genetically modified to express a recombinant antigen receptor or a surface protein are well known in the art. A nucleic acid molecule encoding the antigen receptor or a surface protein may be introduced into the cell in the form of e.g. a vector, or any other suitable nucleic acid construct. Vectors, and their required components, are well known in the art. Nucleic acid molecules encoding antigen receptors can be generated using any method known in the art, e.g. molecular cloning using PCR. Antigen receptor sequences can be modified using commonly-used methods, such as site-directed mutagenesis.

In a particular embodiment, the immune cell is redirected against a cancer antigen. By “cancer antigen” is meant any antigen (i.e. a molecule capable of inducing an immune response) which is associated with cancer. An antigen as defined herein may be any type of molecule which induces an immune response, e.g. it may be a polysaccharide or a lipid, but most preferably it is a peptide (or protein). Human cancer antigens may be human or human-derived. A cancer antigen may be a tumour-specific antigen, by which is meant an antigen which is not found in healthy cells. Tumour-specific antigens generally result from mutations, in particular frame-shift mutations which generate a wholly new amino acid sequence not found in the healthy human proteome.

Cancer antigens also include tumour-associated antigens, which are antigens whose expression or production is associated with (but not limited to) tumour cells. Examples of tumour-associated antigens include for instance Her2, prostate stem cell antigen (PSCA), alpha-fetoprotein (AFP), carcino embryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), CD 19, CD22, CD27, CD30, CD70, GD2 (ganglioside G2), EGFRvIII (epidermal growth factor variant III), sperm protein 17 (Spl7), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ3 (CD61), galactin, K-Ras (V-Ki-ras2 Kirsten rat sarcoma viral oncogene), or Ral-B.

In certain embodiments, the immune cell may be directed against a surface protein selected from CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD15u, CD15s, CD15su, CD16, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85a, CD85d, CD85j, CD85k, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CD156c, CD157, CD158e, CD158i, CD158k, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CD199, CD200, CD201, CD202b, CD203c, CD204, CD205, CD206, CD207, CD208, CD209, CD210, CDw210b, CD212, CD213a1, CD213a2, CD215, CD217a, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240CE, CD240DCE, CD240D, CD241, CD242, CD243, CD244, CD245, CD246, CD247, CD248, CD249, CD252, CD253, CD254, CD256, CD266, CD267, CD268, CD269, CD270, CD271, CD272, CD273, CD274, CD275, CD276, CD277, CD278, CD279, CD280, CD281, CD282, CD283, CD284, CD286, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD308, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, CD371, BCMA, an Immunoglobulin light chain (lambda or kappa), a HLA protein and 02-microglobulin.

In other embodiments, the immune effector cell may be redirected against an antigen associated with an infection, e.g. an antigen from a bacterium, virus, parasite or fungus. The immune cell may alternatively be redirected against an antigen associated with an autoimmune disease or an antigen associated with organ rejection, in particular HLA class I and HLA class II.

In a further aspect, the present invention also provides a pharmaceutical composition comprising CNI-resistant immune cells as described above in combination with one or more pharmaceutically or physiologically acceptable carriers, diluents or excipients. In a particular embodiment, the pharmaceutical composition may further comprise a calcineurin inhibitor as described above such as cyclosporine A, FK506, CTLA-4 Ig and CD28 blocking agent.

The pharmaceutical composition is formulated in a pharmaceutically acceptable carrier according to the route of administration. Preferably, the composition is formulated to be administered by intraveneous injection. Pharmaceutical compositions suitable for such administration may comprise the immune cells, in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

Optionally, the composition comprising immune cells may be frozen for storage at any temperature appropriate for storage of the cells. For example, the cells may be frozen at about −20° C., −80° C. or any other appropriate temperature. Cryogenically frozen cells may be stored in appropriate containers and prepared for storage to reduce risk of cell damage and maximize the likelihood that the cells will survive thawing. Alternatively, the cells may also be maintained at room temperature of refrigerated, e.g. at about 4° C.

The amount of immune cells to be administered may be determined by standard procedure well known by those of ordinary skill in the art. Physiological data of the patient (e.g. age, size, and weight) and type and severity of the disease being treated have to be taken into account to determine the appropriate dosage. The pharmaceutical composition of the invention may be administered as a single dose or in multiple doses. Each unit dosage may contain, for example, from at a dosage of 10⁴ to 10⁹ cells/kg body weight, preferably 10⁵ to 10⁶ cells/kg body weight.

The pharmaceutical composition may further comprise one or several additional active compounds such as corticosteroids, antibiotics, analgesics, immunosuppressants, trophic factors, or any combinations thereof. All the embodiments of the immune cells used according to the invention are also contemplated in this aspect.

According to the present invention, the CNI resistant immune cells according to the invention are used for adoptive cell transfer therapy in a subject undergoing a calcineurin inhibitor treatment. Adoptive cell transfer therapy according to the invention can be used to treat subjects diagnosed with cancer, autoimmune disease, infectious disease, a disease requiring a hematopoietic stem cell transplantation (HSCT) or the prevention of organ rejection.

As used herein, the term “subject”, or “patient” refers to an animal, preferably to a mammal in which an immune response can be elicited including human, pig, chimpanzee, dog, cat, cow, mouse, rabbit or rat. More preferably, the subject is a human, including adult, child and human at the prenatal stage.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

Cancers that may be treated include tumors that are not vascularized, or not yet substantially vascularized, as well as vascularized tumors. The cancers may comprise non solid tumors (such as hematological tumors, for example, leukemias and lymphomas including relapses and treatment-related tumors e.g. secondary malignancies after hematopoietic stem cell transplantation (HSCT)) or may comprise solid tumors. Types of cancers to be treated with immune cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. It can be a treatment in combination with one or more therapies against cancer selected from the group of antibodies therapy, chemotherapy, cytokines therapy, dendritic cell therapy, gene therapy, hormone therapy, laser light therapy and radiation therapy. Of particular interest is posttransplant lymphoproliferative disease (PTLD) that occurs in immunosuppressed patients. For instance, adoptively transferred EBV-specific cytotoxic T lymphocytes (EBV-CTLs) are effective for the treatment of EBV-associated PTLS in solid organ transplant recipients. In addition, other neoplasms are of interest as well since immunosuppressed patients generally have an increased risk for cancer development.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others. In particular embodiment, the autoimmune disease according to the disclosure is an autoimmune disorder treated with immunosuppressive drugs including CNIs and CTLA4-Ig. For example, cellular therapy-based depletion of autoreactive cells including B cells can be achieved using CAR-T or CAAR-T cells in combination with immunosuppressive drugs, graft versus host disease (GvHD) that can occur after allogeneic HSCT can be achieved using cellular T cell therapy, e.g. with Treg, CAR-Treg or other suppressive immune cells in combination with immunosuppression or the pathogenic alloreactive T cells depletion can be achieved using T cell based therapy in combination with immunosuppression.

Infectious disease is a disease caused by pathogenic microorganism such as bacteria, viruses, parasites or fungi. In particular embodiments, infections according to the disclosure occur in immunosuppressed patients, such as patients after HSCT or patients who received a solid organ transplantation. Any disease requiring the use of CNIs or CTLA4-Ig or other immunosuppression predisposes to sometimes serious or lethal infections. T cell-based therapies are promising treatments for lymphopenic and neutropenic patients, e.g. after HSCT. For instance, pathogen-specific, e.g. virus-specific T cells can be enriched or engineered and applied to patients. If the patient concomitantly receives immunosuppressive drugs including CNIs and CTLA4-Ig, rendering the anti-pathogen T cells CNI resistant is particularly important.

Inflammatory diseases are selected from the group consisting of chronic inflammatory disease, chronic inflammatory disease of auto-immune origin, pro-inflammatory and inflammatory conditions in case of cancers.

Hematopoietic stem cell transplantation (HSCT) may be used to treat a number of conditions both congenital and acquired. Acquired disease requiring HSCT include but are not limiting to acute lymphoblastic leukemia (ALL), acute myeloid leukemia (AML), chronic lymphocytic leukemia (CLL), chronic myelogenous leukemia (CML), lymphomas, Hodgkin's disease, non-Hodgkin's lymphoma, multiple myeloma (Kahler's disease), neuroblastoma, desmoplastic small round cell tumor, Ewing's sarcoma, choriocarcinoma, myelodysplasia, paroxysmal nocturnal hemoglobinuria (PNH; severe aplasia), aplastic anemia, acquired pure red cell aplasia, polycythemia vera, essential thrombocytosis, myelofibrosis, amyloidoses, amyloid light chain (AL) amyloidosis, radiation poisoning, HTLV, HIV. Congenital disease requiring HSCT include but are not limiting to lipidoses, neuronal ceroid lipofuscinoses, infantile neuronal ceroid lipofuscinosis, Jansky-Bielschowsky disease, sphingolipidoses, Niemann-Pick disease, Gaucher disease, leukodystrophies, adrenoleukodystrophy, metachromatic leukodystrophy, Krabbe disease, mucopolysaccharidoses, Hurler syndrome, Scheie syndrome, Hurler-Scheie syndrome, Hunter syndrome, iduronidase sulfate deficiency, Sanfilippo syndrome, Morquio syndrome, Maroteaux-Lamy syndrome, Sly syndrome, glycoproteinoses, Mucolipidosis II, Fucosidosis, Aspartylglucosaminuria, Alpha-mannosidosis, Wolman disease, ataxia-telangiectasia, DiGeorge syndrome, severe combined immunodeficiency (SCID), Wiskott-Aldrich syndrome, Kostmann syndrome, Shwachman-Diamond syndrome, Griscelli syndrome, type II, NF-Kappa-B Essential Modulator (NEMO) deficiency, Sickle cell disease, β thalassemia major, aplastic anemia, Diamond-Blackfan anemia, Fanconi anemia, cytopenias, amegakaryocytic thrombocytopenia, hemophagocytic lymphohistiocytosis (HLH).

The term “organ rejection” refers to a state in which a transplanted organ or tissue is not accepted by the body of the recipient. Rejection results from the recipient's immune system attacking the transplanted organ or tissue. Rejection can occur days to weeks after transplantation (acute) or months to years after transplantation (chronic). Cellular T cell therapy, e.g. with Treg, CAR-Treg or other suppressive immune cells to prevent or treat organ rejection in combination with immunosuppression is particularly interesting. Patients receiving allogenic solid organ grafts can be rendered tolerant if concomitantly transplanted with HSCs from the same allogeneic donor. However, this therapy bears the risk for GvHD and is accompanied by immunosuppressive drugs.

In another embodiment, the present invention relates to a method for treating cancer, autoimmune disease, an infectious disease or for the prevention of organ rejection in a subject in need thereof, comprising administrating a therapeutically efficient amount of CNI-resistant immune cell in which the regulatory activity of miR-17˜92 cluster or paralogs thereof is increased, preferably CNI-resistant immune cell engineered to overexpress at least one miRNA of the miR-17˜92 cluster or paralogs thereof, or a pharmaceutical composition of the invention in combination with (e.g., before, simultaneously or following) a calcineurin inhibitor as immunosuppressive agent such as Cyclosporin A, FK506, also named Tacrolimus or CTLA4-Ig.

By a “therapeutically efficient amount” is intended an amount of CNI-resistant immune cells administered to a subject that is sufficient to constitute a treatment as defined above.

In a particular embodiment, immune cells to be utilized in the ACT therapy are previously cultured in presence of CNI to select CNI-resistant immune cells in which regulatory activity of the miR-17˜92 cluster or paralogs thereof is increased. In the presence of CNI, only CNI-resistant immune cells in which regulatory activity of the miR-17˜92 cluster or paralogs thereof, preferably engineered as described above to overexpress at least one miRNA of the miR-17˜92 cluster or paralogs thereof or engineered to inactivate or repress the expression of at least one miR-1792 cluster target gene. Thus, the present disclosure relates to a method of preparing CNI-resistant immune cell as described previously comprising the step of: i) engineering an immune cell to overexpress at least one miRNA of miR-17˜92 cluster or paralogs thereof target gene or to inactivate the expression of at least one miR-17˜92 cluster target gene as described above ii) culturing said immune cells in presence of CNI, iii) selecting CNI-resistant immune cells.

Optionally, CNI resistance can be removed after the step of selection, for example by inactivating the expression of at least one miRNA of the miR-17˜92 cluster, or by expressing at least one miR-1792 cluster target gene. In this particular case, CNI resistant conferred to the cells is used as a reporter gene to select correctly targeted cells ex vivo, preferably before to be utilized in the ACT therapy.

In another particular embodiment, said CNI-resistant immune cells can therefore be utilized in the ACT therapy in a subject in need thereof. Thus, in a particular embodiment, the present disclosure relates to a method for treating cancer, autoimmune disease, an infectious disease or for the prevention of organ rejection in a subject in need thereof, comprising: i) preparing CNI-resistant immune cells as described previously and ii) administrating a therapeutically efficient amount of CNI-resistant immune cell in combination with (e.g., before, simultaneously or following) a calcineurin inhibitor as immunosuppressive agent such as Cyclosporin A, FK506, also named Tacrolimus or CTLA4-Ig.

The administration of the immune cells or pharmaceutical composition according to the present invention may be carried out in any convenient manner, including by aerosol inhalation, injection, ingestion, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the immune cells or pharmaceutical compositions of the present invention are preferably administered by intravenous injection. The immune cells or pharmaceutical compositions of the present invention may be injected directly into a tumor, lymph node, or site of infection.

The administration of the cells or population of cells can consist of the administration of 10⁴-10⁹ cells per kg body weight, preferably 10⁵ to 10⁷ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In another embodiment, said effective amount of cells or composition comprising those cells are administrated parenterally.

CNI resistant immune cells or pharmaceutical compositions are administered to a subject in combination with (e.g., before, simultaneously or following) a calcineurin inhibitor as immunosuppressive agent such as Cyclosporin A or FK506, also named Tacrolimus or CTLA4-Ig.

In certain embodiments of the present invention, the CNI-resistant immune cells according to the invention can also be administered to a subject in combination (e.g., before, simultaneously or following) with an immune-checkpoint pathway inhibitor such as PD-1 inhibitor, PDL-1 inhibitor or a CTLA-4 inhibitor, preferably PD-1 inhibitor, in particular an anti-PD1 antibody.

The following examples are given for purposes of illustration and not by way of limitation.

### Examples

1. Material and methods

1.1 Mice

C57BL/6-Gt(ROSA)26Sortm3(CAG-MIR17˜92,−EGFP)Rsky/J (miR1792tg) (Xiao, C., et al., Nat Immunol, 2008. 9(4):405-14) and Mirc1tm1.1Tyj/J (miR1792lox) (Ventura, A., et al., Cell, 2008. 132(5):875-86) mice were bred to B6.Cg-Tg(Cd4-cre)1Cwi/BfluJ (CD4cre) (Lee, P. P., et al. Immunity, 2001. 15(5):763-74). Cre negative littermates served as wt controls. The B6.CD4cre.R26miR1792tg strain was additionally bred to Cd28ko mice (Shahinian, A., et al. Science, 1993. 261(5121):609-12) for the rescue model, here cre negative littermates were used as CD28ko. Male and Female mice were used at 5-6 weeks of age at the starting day of the experiment. Animals were maintained under specific pathogen free conditions in accordance with institutional guidelines at Department of Biomedicine of the University Hospital Basel.

1.2 Organ and Blood Isolation

Organs were obtained after CO₂ euthanization and kept on ice during processing. Mesenteric lymph nodes (LN), inguinal LN, axillary LN, brachial LN, cervical LN and spleen were taken for most of the experiments. Spleens were injected with 0.5 ml ACK lysis buffer for erythrolysis before processing. The organs were meshed with 0.4 μm filters to obtain single cell suspensions which were then washed with FACS buffer. Blood for ELISA was taken immediately after euthanisation with heart puncture. The blood was incubated at RT for 30 min in wax tubes and then centrifuged for 10 min, 7000 rpm to separate the serum. Serum was then transferred to fresh tubes and frozen at −20° C. until analysis.

1.3 Naïve CD4 T Cell Isolation

Naïve CD4 T cells were isolated from cell suspensions with pooled lymph nodes and spleen. Isolation was performed with StemCell mouse naïve CD4 T cell isolation kit according to manufacturer's instructions. The resulting untouched naïve CD4 T cells were then washed with FACS buffer, and purity was routinely checked with a staining for CD4, CD44 and viability.

1.4 Proliferation Assay with Cell Trace Violet (CTV)

Freshly isolated naïve CD4 T cells were washed with PBS. 1 μl of Cell Trace stock solution (dissolved in DMSO according to the manufacturer's instructions) was then used per ml PBS for 10*10⁶ cells. Cells were incubated at 37° C. for 20 min, then 5× the original staining volume of normal T cell culture medium was added for 5 min to remove residual dye. Cells were then washed again and plated in complete culture medium.

1.5 Plate-Bound CD4 T Cell Activation

Plates were coated overnight with 0.2 μg αCD28 and 0.5 μg αCD3 per ml PBS for most of the experiments (low stimulation (Baumjohann, D., et al. Nat Immunol, 2013. 14(8):840-8)). Plates were washed with PBS before plating 2*10⁵ cells per well in 96 well flat-bottom in 200 μl medium with 50 U IL-2 per ml. For 24 well plates, 2*10⁶ cells per ml medium were plated.

1.6 In Vitro Differentiation

TH1 differentiation conditions were generated with 50 U IL-2, 5 ng/ml IL12 and 10 μg/ml αIL4 per ml T cell medium. TH2 differentiation conditions were reached with 50 U IL-2, 50 ng/ml IL4, 5 μg/ml αIFNγ and 10 μg/ml αIL12/IL23. iTregs were either differentiated with or without retinoic acid (0.9 mM), but always with 250 U IL-2, 0.75 ng/ml TGFβ, 10 μg/ml αIFNγ and 10 μg/ml αIL4.

TH17 were generated with 50 ng/ml IL6, 3 ng/ml TGFβ, 5 μg/ml αIFNγ and 10 μg/ml αIL4 per ml T cell medium. For the differentiation, 2*10⁵ naïve CD4 T cells were plated on a pre-coated 96-well flat bottom plate (coated over night with 0.2 μg αCD28, 0.5 μg αCD3 per ml PBS) and harvested at 24 h, 48 h or 72 h after plating. T cell subsets were then stained for hallmark cytokine/transcription factor combinations: TH1 (Tbet/IFNγ), TH17 (Rorγt/IL17A) and iTreg (FoxP3/CD25).

1.7 Seahorse

Calibration plates were coated overnight with 200 μl calibrant. Cell plates were coated with 18 μl 0.1M NaHCO3 pH8.0 6.67% CellTak (Seahorse XF96 flux pack, Bucher Biotech, CH). The following day, cell plates were washed with H₂O and left for drying during cell preparation. Compounds were prepared for a final in-well concentration of 1 μM for Oligomycin, 2 μM for FCCP and 11 μM for Rotenone. Cells were prepared in glucose-free RPMI, washed and counted multiple times before plating 3*10⁵ cells per well in glucose-free RPMI. Mitochondrial perturbation was performed by sequential injection of glucose (80 mM stock), oligomycin, FCCP and rotenone. Measurements of oxygen consumption rate (OCR, pMoles/min) and extracellular acidification rate (ECAR; mpH/min) were performed with a Seahorse XF96 flux analyzer (Seahorse Bioscience, USA). Data analysis was performed using Prism (Version7.0d), mitochondrial parameters were calculated as described by Gubser et al. (Gubser, P. M., et al. Nat Immunol, 2013. 14(10): p. 1064-72).

1.8 FACS Staining

For cytokine stainings cells were stimulated with 50 ng/ml PMA, 500 ng/ml Ionomycin and 10 μg/ml BFA for 3 h at 37° C. before staining (GP-64 instead of PMA/Iono in LCMV experiments, see section LCMV). Cells were then first stained for viability with viability dye 780 in PBS for 20 min at 4° C. and then washed with PBS. Non-specific binding was blocked with αCD16/αCD32 0.5 mg/ml on ice for 10 min. Surface stainings were performed in FACS buffer for 20-30 min at 4° C. Cell fixation was performed with Fix-Perm for at least 20 min on 4° C. (1 h for LCMV experiments). Intracellular staining was done in Permeabilisation buffer for 1 h at 4° C. Data was acquired with Fortessa and analyzed with FlowJo (version 10.4.1)

1.9 RNA Extraction for qPCR

Cells were washed with PBS before counting and the RNA was kept on ice. 5*10⁵ cells were then resuspended in 400 μl TRIreagent. RNA isolation was then performed according to the isolation protocol from TRIreagent supplier (SIGMA). In brief, 0.1 ml of 1-bromo-3-chloropropane per ml of TRI Reagent was added, samples were mixed, incubated for 15 min at room temperature and then centrifuged at 12′000 g for 15 min at 4° C. for phase separation. The aqueous phase was then mixed with 0.5 ml of isopropanol per ml of TRI Reagent used, again centrifuged for 10 min for RNA precipitation. RNA was then washed with 75% Ethanol and finally resuspend in RNAse-free water. RNA concentration and purity was afterwards determined with Nanodrop.

1.10 RNA Extraction for RNA Sequencing, Protein Extraction and Digestion for Proteomics

All procedures for the extractions were performed at the facilities with materials, protocols and supervision of the facility experts. RNA for the sequencing was extracted from Trizol-samples with a Zymo Direct-zol kit which includes DNAse treatment. Quality control was run with a Bioanalyzer. Proteins were extracted and digested for mass spectrometry with Lys-C and Trypsin.

1.11 Reverse Transcription and Quantitative PCR

For Rcan3 qPCR, cDNA was generated with the SIGMA MMLV kit on 1 μg RNA. qPCR was run with 18S as a reference for the experiments looking at Rcan3. qPCR was then run with TaqMan FAST Universal PCR master mix on an Applied Biosystems® Real-Time PCR System.

1.12 Glucose Uptake Staining with 2-NBDG

2*10⁵ naïve CD4 T cells were washed with medium and then incubated with medium plus 50 μM 2-NBDG for 30 min at 37° c. 5× the original staining volume of normal T cell culture medium was then added for 5 min to remove residual dye. Cells were then washed again and stained for viability and surface markers before FACS analysis.

1.13 ELISA

IL-2 ELISA was performed with the BioLegend ELISA MAX mouse IL-2 set according to the manufacturer's instructions.

1.14 LCMV Armstrong Experiments

Mice were infected with 2*10⁵ PFU LCMV-Armstrong strain intra peritoneally. Virus was provided by the Recher Laboratory. Eight days post infection, the animals were euthanized with CO2 and the spleens were harvested. Re-stimulation of splenocytes was performed in flat bottom 96 well plates with LCMV-specific peptide 1 μg/ml GP-64 in comparison to polyclonal 50 ng/ml PMA, 500 ng/ml Ionomycin stimulation for one hour, then 10 μg/ml Brefeldin A was added for another three hours before staining.

1.15 Cyclosporin A Titration

Increasing amounts of calcineurin inhibitors, Cyclosporin A or FK506 were added to the cultures: 2*10⁵ cells were plated in 100 μl complete T cell medium in pre-coated 96 well plates. 100 μl of a serial dilution of calcineurin inhibitors were then added to result in in-well at different concentrations. Cells were then activated in the presence of these calcineurin inhibitors concentrations for 48 h before harvesting and staining for viability and activation markers.

1.16 Imagestream

Naïve CD4 T cells were isolated and activated for 48 h as described before. They were then harvested and washed with PBS before fixation for 20 min at 4° C. Intracellular staining for NFATc2 was performed in a two-step staining with first 1 h at room temperature (RT) with aNFATc2 in permeabilization buffer, and subsequently 1 h at room temperature (RT) with goat anti-mouse IgG1 in permeabilization buffer. Nuclei were stained with DAPI in the last 5 min of incubation. Acquisition was run on a ImageStreamX Mark2 Imaging flow cytometer, and data analysis was performed with the IDEAS software.

2. Results

2.1 miR-17˜92 Influences Downstream Events of CD4 T Cell Activation and Expansion

CD28 co-stimulation is essential for CD4 T cell function including e.g. cytokine production (Sanchez-Lockhart, M., et al. J Immunol, 2004. 173(12):7120-4), proliferation (Levine, B. L., et al. 1997. 159(12):5921-30), germinal center formation (Wang, C. J., et al. Proc Natl Acad Sci USA, 2015. 112(2):524-9) and glycolytic switch (Frauwirth, K. A., et al. Immunity, 2002. 16(6):769-77). It also induces miR-17˜92 expression (de Kouchkovsky, D., et al. 2013. 191(4):1594-605), which in turn was reported to influence similar processes (Baumjohann, D., et al. Nat Immunol, 2013. 14(8):840-8; Xiao, C., et al. Nat Immunol, 2008. 9(4):405-14).

To directly compare how CD4 T cells lacking or overexpressing miR-17˜92 react on activation, the inventors took advantage of previously published mouse models CD4cre.miR1792lox (miR1792lox) and CD4cre.R26miR1792tg (miR1792tg) (Xiao, C., et al. Nat Immunol, 2008. 9(4):405-14; Ventura, A., et al. Cell, 2008. 132(5):875-86; Lee, P. P., et al. Immunity, 2001. 15(5):763-74) and used CD4cre negative littermates as wt controls. Naïve CD4 T cells from spleen, peripheral and mesenteric lymph nodes were isolated and used for subsequent activation.

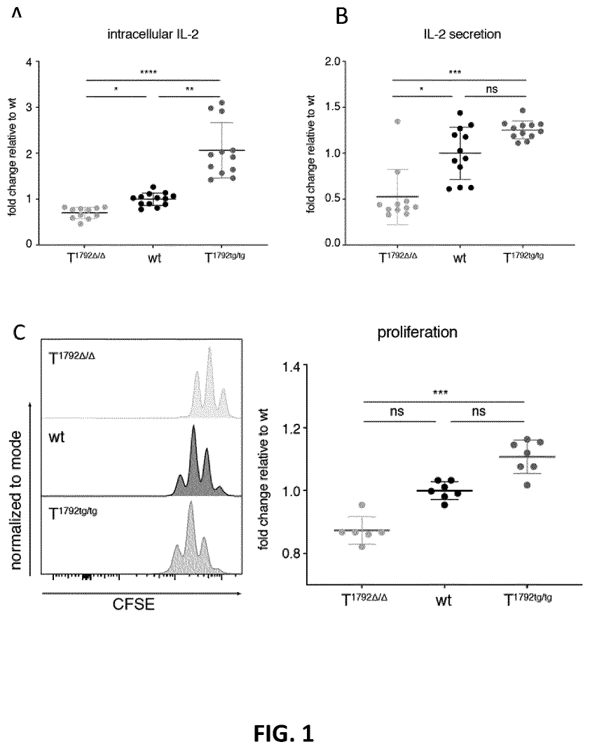

Upon 3 h activation with PMA/Ionomycin/BFA the inventors found a ˜1.7-fold increase in IL-2 production with transgenic miR-17˜92 expression (FIG. 1A), and less staining in miR1792lox cells. This was also true for IL-2 secretion measured by ELISA in culture supernatants of T cell cells that were activated for 48 h (FIG. 1B): miR1792lox cells secreted only half of the IL-2 that was secreted by wt cells. The proliferation capacity was proportional to the cluster expression (FIG. 1C), which is consistent with previous reports (Baumjohann, D., et al. Nat Immunol, 2013. 14(8):840-8).

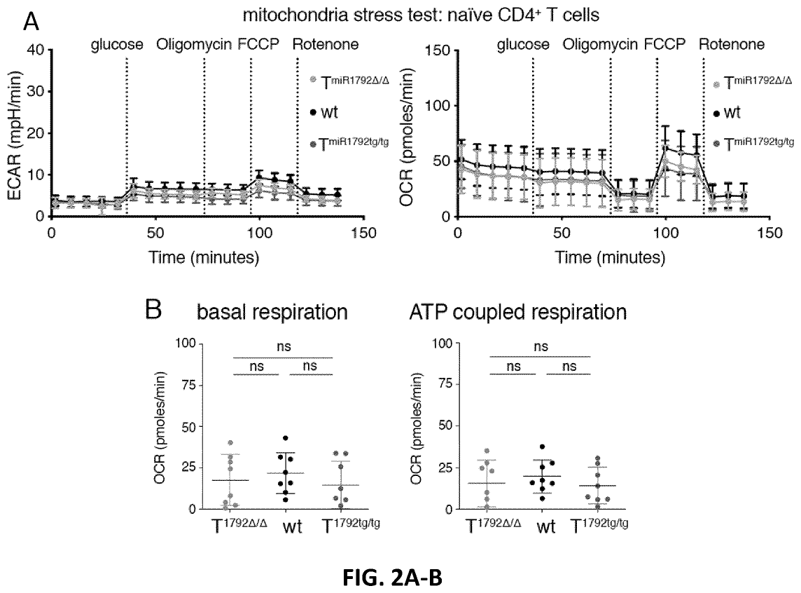

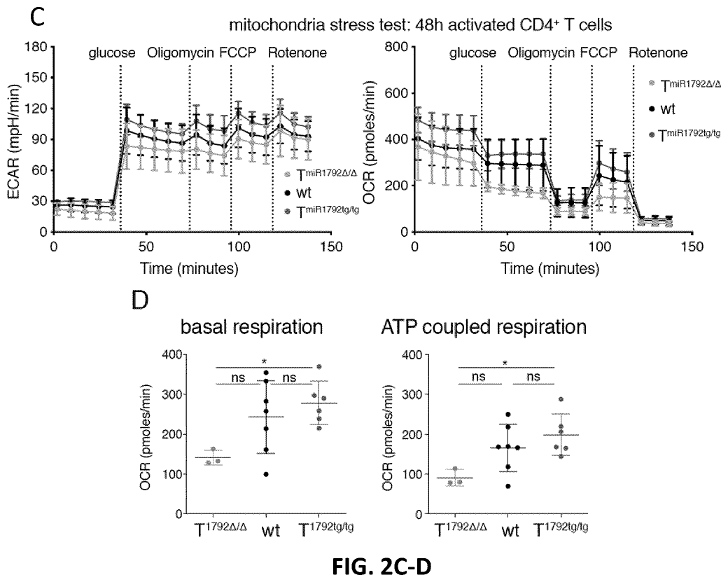

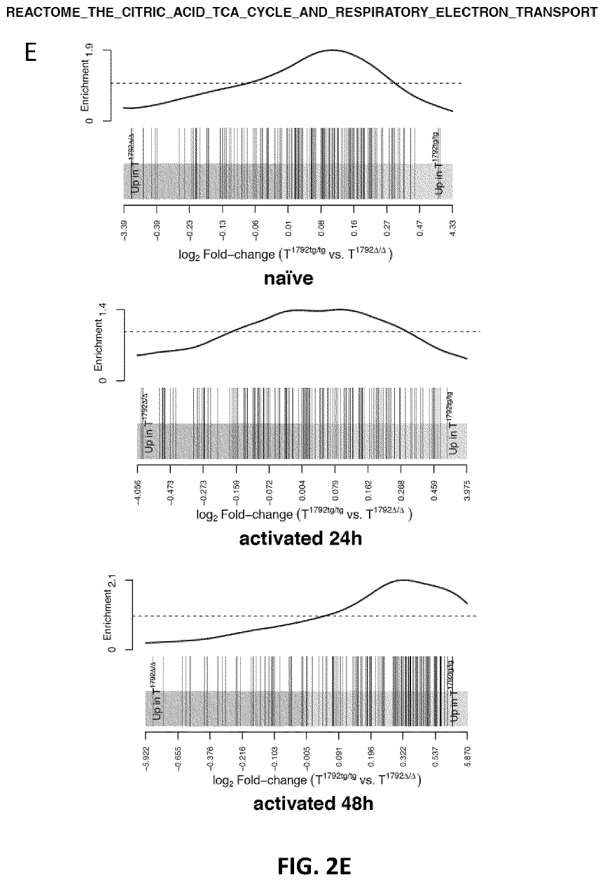

Metabolic activity of naïve cells was similar among genotypes (FIG. 2A). In contrast, activated CD4 T cells from miR1792lox mice had a slightly reduced activity in their metabolism (FIGS. 2C and D). Additionally, the inventors analyzed the metabolome of the cells with metabolomics and found that there is a slight increase in the abundance of certain metabolites in miR1792tg cells (data not shown), but no specific pathway stood out. The inventors furthermore investigated RNA sequencing data for genes that are associated with expression of metabolic pathways and detected a slightly higher expression of genes involved in TCA and electron transport chain in miR1792tg cells as compared to wt (FIG. 2E). From this data the inventors concluded that the microRNA cluster miR-17˜92 influences processes that are important in T cell activation, and increased miR-17˜92 expression led to a “more activated” cell phenotype, which consequently also leads to a more active metabolic phenotype.

2.2 Transgenic miR-17˜92 Expression Rescues CD28 Deficiency In Vitro

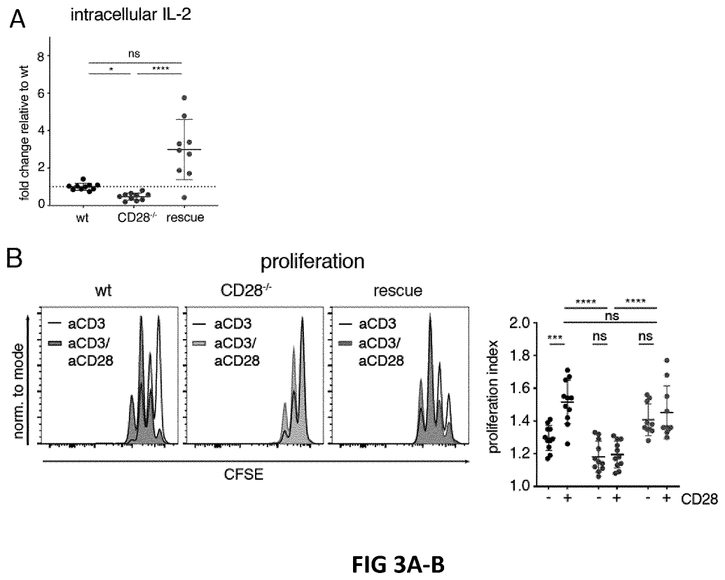

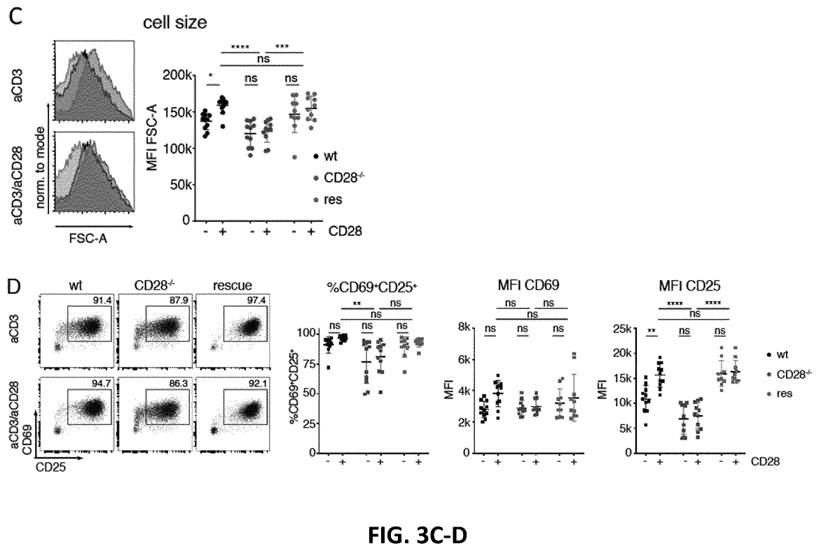

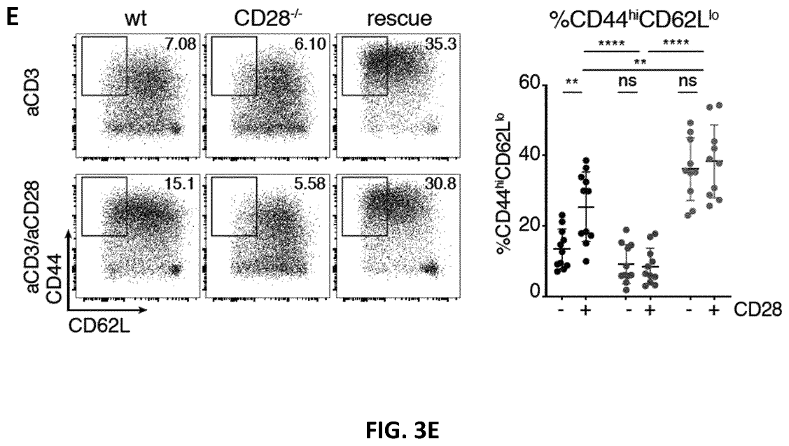

Since depleting miR-17˜92 expression led to a similar phenotype as reduced CD28 signalling, the inventors hypothesized that transgenic expression of miR-17˜92 might be able to partially rescue the phenotype of CD28ko cells. They crossed CD4cre.R26miR1792tg to CD28ko mice (Shahinian, A., et al. Science, 1993. 261(5121):609-12) to receive CD4cre.R26miR1792tg.CD28ko mice (rescue) and isolated naïve CD4 T cells like before. CD28ko CD4 T cells produced half of the IL-2 amount that was seen in wt cells upon 3 h PMA/Ionomycin stimulation, while rescue cells produced ˜2.5 fold more than wt (FIG. 3A). The inventors then investigated the behaviour of the CD4 T cells after in vitro activation with αCD3 for 48 h with or without a CD28 co-stimulation. The proliferation capacity was reduced in activation without αCD28 co-stimulation but rescued with transgenic miR-17˜92 expression (FIG. 3B). The size of the blasting cells, shown as FSC-A (FIG. 3C), was reduced with missing CD28 signal but rescued with transgenic miR-17˜92 expression. After 48 h activation (FIG. 3D) with or without αCD28 co-stimulation, all genotypes developed similar CD69⁺CD25⁺ populations.

However, the MFI of CD25 was dependent on CD28 signalling, and rescued by transgenic miR-17˜92 expression. As opposed to this, CD69 expression which is known to be dependent on TCR signaling (Testi, R., J. H. Phillips, and L. L. Lanier. J Immunol, 1989, 142(6):1854-60) was indifferent among all conditions. Also for late activation marker CD44 (FIG. 3E) the inventors observed a strong dependency on CD28 or miR-17˜92 signalling.

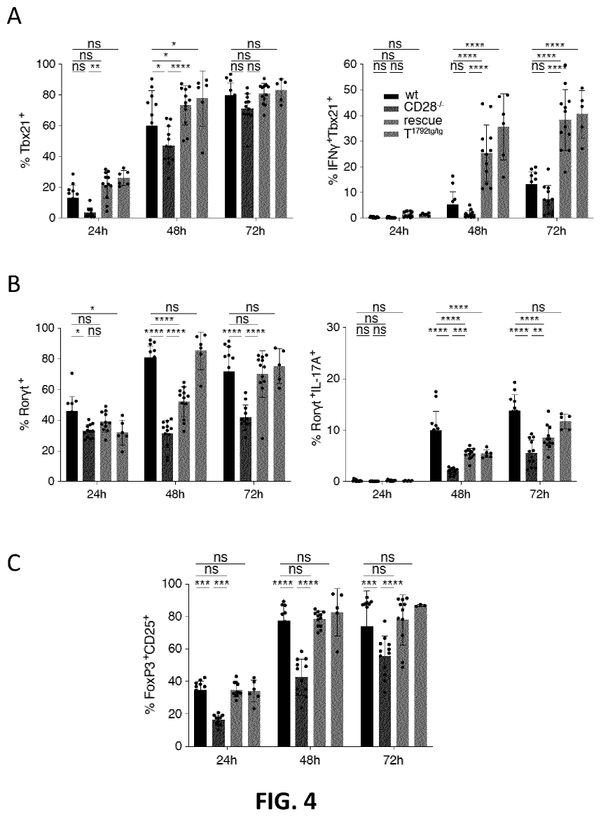

The miR-17˜92 cluster was reported to influence the differentiation of TH1 (Wu, T., et al. J Immunol, 2015. 195(6):2515-9; Jiang, S., et al. Blood, 2011. 118(20):5487-97), TH2 (Simpson, L. J., et al. Nat Immunol, 2014. 15(12):1162-70), TH17 (Liu, S. Q., et al. J Biol Chem, 2014. 289(18):12446-56) and Tregs. The inventors therefore performed differentiation assays with CD28ko and rescue cells and looked at TH1, TH17 and iTreg skewing (FIG. 4). Tbet (Tbx21) induction was delayed in CD28ko cells but rescued with miR-17˜92 expression, and IFNγ production was even over compensated by transgenic miR-17˜92 expression (FIG. 4A). Reduced Rorγt induction and IL17a production in CD28ko cells was partially rescued (FIG. 4B). iTreg induction led to a reduced population of FoxP3+CD25+ population in the CD28ko sample in all time points which was rescued to wt levels (FIG. 4C). Overall, all tested T cell subsets were affected by CD28ko and rescued to some extent by transgenic miR-17˜92 expression. However, timing and extent of rescue varied between subsets, which argues that the relative contribution is also variable.

Collectively, these data demonstrate known defects in CD4 T cell activation in CD28ko cells could be rescued by transgenic miR-17˜92 expression in vitro. Moreover, the rescue effect observed in in vitro differentiation assays suggested a mechanism upstream of the differentiation-decision pathways.

2.3 Transgenic miR-17˜92 Expression Rescues CD28 Deficiency In Vivo

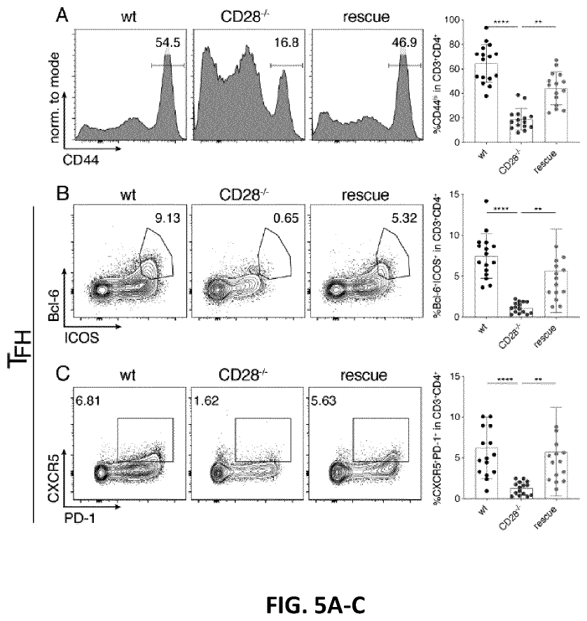

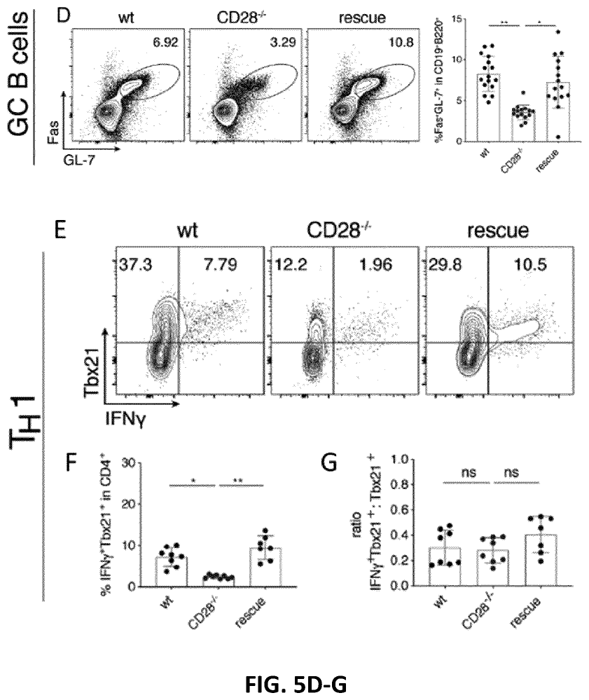

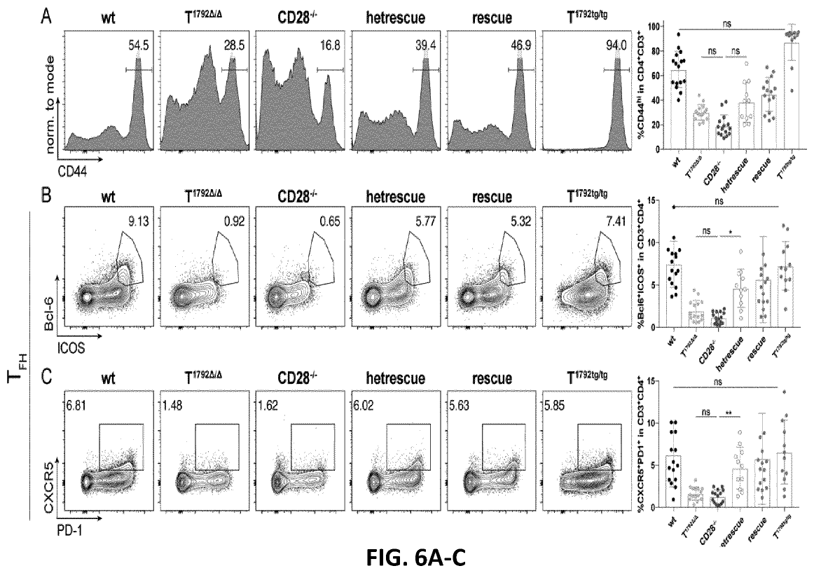

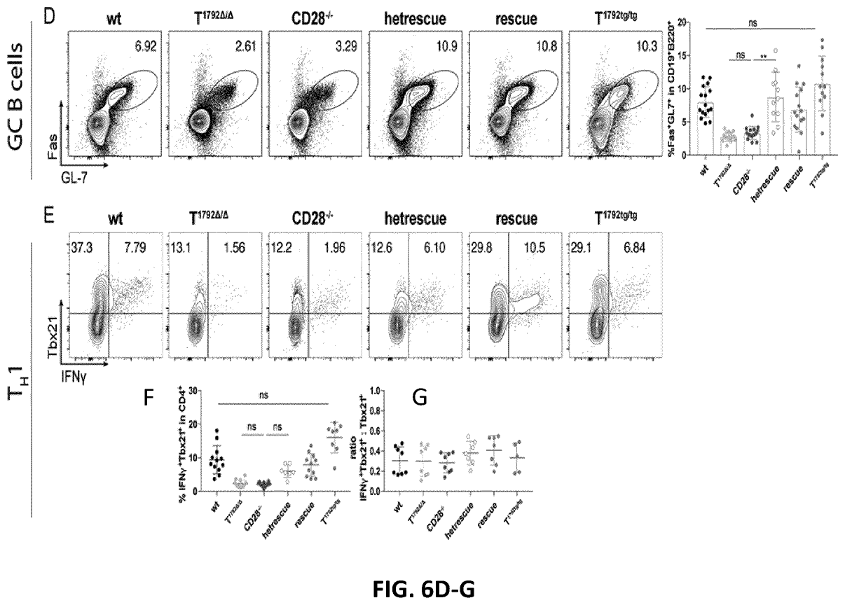

To investigate the generation of germinal center (GC) and TH1 responses in vivo, the inventors infected the rescue mouse model with LCMV Armstrong and analyzed the spleen 8 days post infection. In line with in vitro data the inventors observed a decreased CD44 expression in CD28ko CD4 T cells, which was rescued by transgenic miR-17˜92 expression (FIG. 5A). TFH, stained with key markers Bcl6, ICOS (FIG. 5B), CXCR5 and PD-1 (FIG. 5C), showed a 5-fold reduced population in CD28 mice, which was rescued by transgenic miR-17˜92. The comparison of miR1792lox to CD28ko mice (FIG. 6) demonstrated the same phenotype. GL-7⁺Fas⁺ germinal center (GC) B cells, which are known to be absent when the CD28 co-stimulatory signal is missing (Wang, C. J., et al., Proc Natl Acad Sci USA, 2015. 112(2):524-9), was rescued with transgenic miR-17˜92 expression (FIG. 5D). Upon restimulation with GP-64 (Oxenius, A., et al. Eur J Immunol, 1995. 25(12):3402-11; Wolint, P., et al. J Exp Med, 2004. 199(7):925-36), Tbet (Tbx21) expression was 2-fold reduced in CD28ko cells but not significantly increased in rescue cells. However, IFNγ production was 5-fold reduced in CD28ko and fully restored by transgenic miR-17˜92 expression. The ratio of IFNγ producing cells to total Tbet expressing cells was indifferent among all genotypes, which argues for a block at transcription factor but not at cytokine production level (FIG. 5E-G).

Taken together, also in vivo transgenic miR-17˜92 expression rescues CD28ko cells. While homozygous expression of the transgene rescued TFH and GC as well as TH1 formation, heterozygous expression was also sufficient for the rescue of TFH and GC B cells but not for TH1 formation (FIG. 6).

2.4 Restoration of CD28 Function by miR-17˜92 is Cell Intrinsic

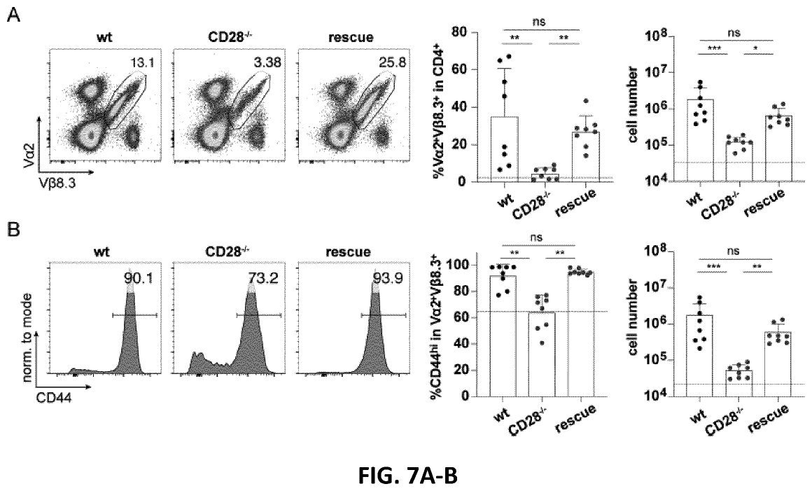

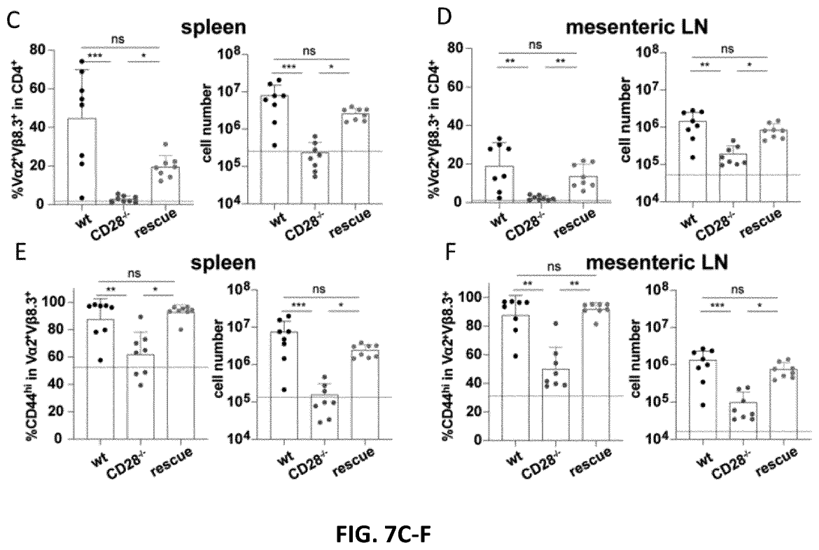

Although CD28's main function is on T cells, the inventors sought to formally test whether the miR-17˜92-mediated rescue effect was cell intrinsic. The inventors crossed MHC class II restricted CD4⁺ TCR transgenic mice specific for LCMV (SMARTA, Vα2+Vβ8.3+) to wt, CD28^{-/-} and rescue mice. The inventors adoptively transferred (AT) naïve CD4⁺ T cells to CD28^{-/-} host mice followed by acute LCMV infection. Eight days post infection, the inventors isolated spleen, mesenteric and peripheral lymph nodes (LN). In all three organs the frequency and absolute number of Vα2⁺Vβ8.3⁺ CD28^{-/-} cells was strongly reduced compared to Vα2+Vβ8.3+CD28^{wt/wt} cells. In contrast, the miR-17˜92 transgene restored relative and absolute numbers of Vα2⁺Vβ8.3⁺ CD28^{-/-} T cells (FIGS. 7A, C and D)). Furthermore, among Vα2⁺Vβ8.3⁺ T cells, fewer CD28^{-/-} cells upregulated CD44 than in wt cells, a defect that was entirely restored in rescue cells (FIGS. 7B, E and F). Thus, these data unequivocally demonstrate that transgenic miR-17˜92 cell intrinsically substituted CD28-deficiency. Collectively, the inventors conclude that the non-coding RNA miR-17˜92 can mediate costimulatory function.

2.5 miR-17˜92 Under- or Overexpression Induces Transcriptional Changes that are Important in T Cell Activation

As the inventors observed a strong effect of miR-17˜92 expression on processes of T cell activation, they were interested in the target genes of this miRNA cluster that might explain for these phenomena. So far, none of the known target genes, like e.g. PTEN or Rorα, could fully explain for the reported phenotypes. Naïve CD4 T cells from miR1792lox, wt and miR1792tg mice were activated for 24 h or 48 h to then isolate total RNA for sequencing.

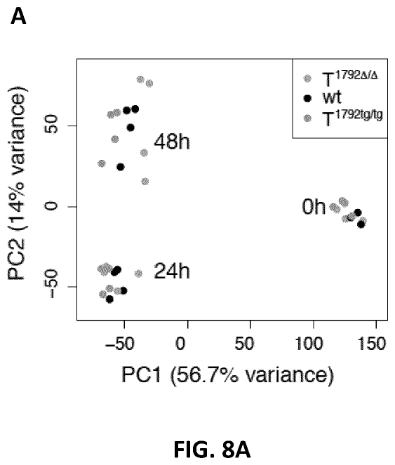

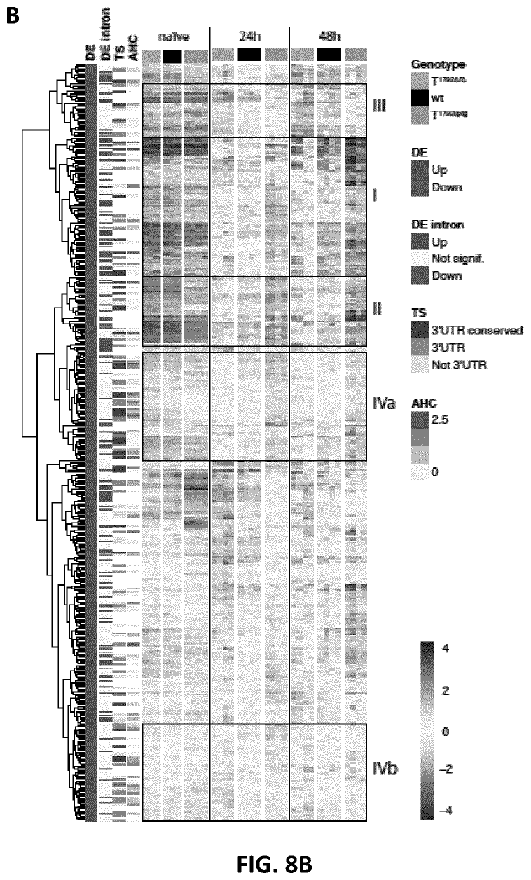

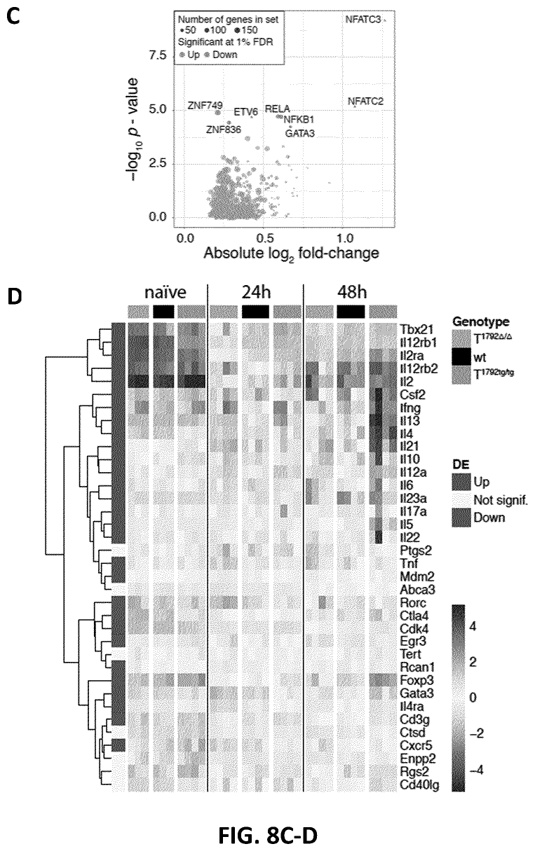

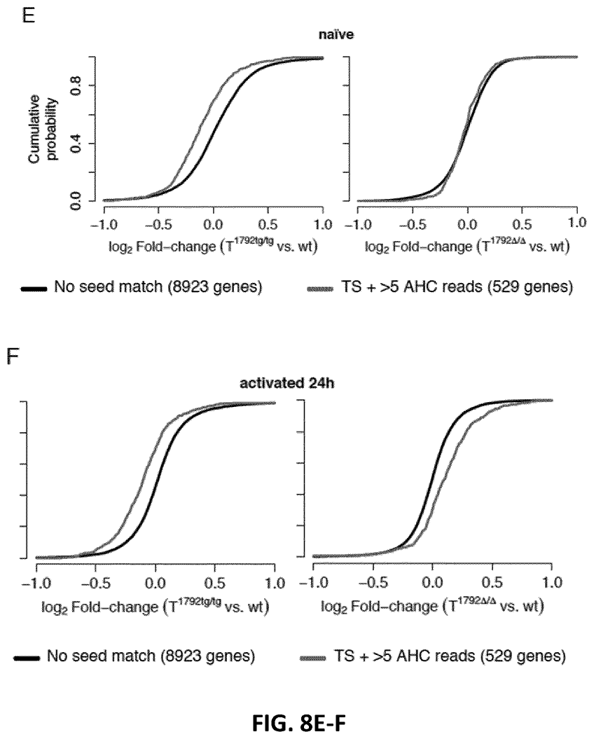

Principal component analysis (PCA) revealed that the transcriptomes of naïve T cells from all three genotypes were very similar, particularly for the wt and T^{1792Δ/Δ} genotypes (FIG. 8A). T cell activation induced major changes in gene expression (PC1, 56.7% of variance explained and PC2, 14% of variance explained) and also made the genotypes separate at 24 h and even more clearly at 48 h after activation (FIG. 8A) (PC1 and PC3, 7.2% of variance explained). Since miRNAs often repress individual genes only mildly (Bartel, D. P. et al. Cell, 2018. 173(1):20-51), the inventors compared the most extreme genotypes, i.e. T^{1792Δ/Δ} to T^{1792tg/tg} to increase the power of differential gene expression analysis, at each time point. At a false discovery rate (FDR) of 1%, the number of differentially expressed genes (DEG) increased over time (830 genes up-regulated and 789 genes down-regulated at 0 h, 2,493 up and 2,370 down at 24 h, and 3,173 up and 3,242 down at 48 h). Unsupervised hierarchical clustering of DEG 24 h after activation revealed a nuanced pattern of gene clusters (FIG. 8B). As expected from the PCA (FIG. 8A), gene expression across genotypes was very similar in naïve T cells and the magnitude of expression differences increased after activation (FIG. 8B). According to their expression profile, the inventors defined 4 different groups of genes (FIG. 8B): cluster I genes were induced over time and enhanced in T^{1792tg/tg} compared to wt but reduced or delayed in T^{1792Δ/Δ}. Cluster II genes decreased with time and miR-17˜92 supported their repression. Overall cluster III gene expression increased after activation but expression per time point inversely correlated with the genotype (T^{1792Δ/Δ}>T^{1792tg/tg}). Thus, miR-17˜92 limited initial or final maximal expression of genes in this group despite their induction with time. Finally, genes displaying the most obvious inverse correlation with the genotype were grouped in clusters IVa and IVb. To summarize, cluster I most likely represents indirectly regulated genes while clusters IVa and IVb most likely include the majority of direct miR-17˜92 target genes.

To analyze gene regulation modalities of the different clusters, the inventors used an exon-intron split analysis (EISA) approach whose aim is to discriminate between transcriptional versus posttranscriptional regulation. This method relies on differences between mature and pre-mRNA intronic and exonic read counts. In RNA-seq experiments these changes are good predictors of transcriptional rate changes (Gaidatzis D. L. et al. Nat Biotechnol, 2015. 33(7):722-9). In addition, the inventors employed computational target gene predictions for each seed family of the miR-17˜92 cluster from the Targetscan (“TS”) database (Agarwal V. et al. Elife, 2015.33(7):722-9). Targetscan allowed the identification of evolutionary conserved 8mer, 7mer and 6mer corresponding to miRNA seed families. Furthermore, the inventors used a dataset of biochemically detected direct miRNA/mRNA interactions in T cells defined by Argonaute 2 high-throughput sequencing of RNA isolated by crosslinking immunoprecipitation (“AHC”) (Gagnon J. D. et al. Cell Rep, 2019,28(8):2169-2181) and quantified for each gene the read coverage on TS seed matches for each of the miR-17˜92 cluster seed families. These predictors of transcriptional (“DE.intron”) and posttranscriptional regulation (“TS”, “AHC”), annotated on the heatmap (FIG. 8B), revealed genes whose expression increased over time and positively correlated with the miR-17˜92 genotype. They were enriched for transcriptional regulation and displayed few TS sites or AHC reads (FIG. 8B, box I). Thus, cluster I was mainly induced by increased gene transcription and miR-17˜92 promoted this transcriptional activity. In contrast, genes from clusters IVa and IVb (FIG. 8B, boxes IVa, IVb) displayed a consistent and colinear reduction of expression levels across genotypes that inversely correlated with miR-17˜92 dosage (T^{1792Δ/Δ}>wt>T^{1792tg/tg}) at 24 h and 48 h. Importantly, these clusters contained few transcriptionally regulated genes but were enriched for TS sites (p-value=0.001037; Fisher test) and experimentally determined AHC reads (p-value=0.003598; Fisher test) (FIG. 8B). Thus, these clusters were mainly regulated posttranscriptionally and enriched for empirically validated direct miR-17˜92 target genes. In summary, miR-17˜92 became functionally relevant after T cell activation and shaped the transcriptome in intricate ways. The inventors concluded that miR-17˜92 can indirectly promote gene induction, support gene silencing indirectly and through direct inhibition or dampen expression of induced genes.

Direct target candidates of the miR-17˜92 cluster should be down-regulated the more cluster is expressed and have a binding site for at least one member of the miR-17˜92 cluster. To further narrow down the list of interesting genes the inventors compared the data to data sets from Ago-HITSCLIP RNA sequencing. This method reveals genes whose 3′UTR are bound by Argonaute at the moment of sequencing, so genes that are targeted for their degradation by the RISC complex. The comparison confirmed that the genes with a seed match in their 3′UTR as predicted by Targetscan do get downregulated in inventors dataset and also in the HITS-CLIP dataset.

In the gene set enrichment analysis of the RNA sequencing the inventors noted an enrichment for gene sets that were associated with cytokine expression in the top 25 significantly changed gene sets (See Table 4).

TABLE 4
Top 25 most significant “curated gene sets” from KEGG/Biocarta/Reactome in the
comparison miR1792lox vs. miR1792tg. Shown in grey are the gene sets associated with cytokine
expression.

| GeneSet | NGenes | Direction | absLog2FC | PValue | adj.P.Val |
|---|---|---|---|---|---|
| KEGG_ALLOGRAFT_REJECTION | 38 | Up | 0.86425538 | 1.16E-08 | 2.30E-05 |
| KEGG_TYPE_I_DIABETES_MELLITUS | 39 | Up | 0.85594368 | 2.09E-08 | 2.76E-05 |
| REACTOME_INTERFERON_GAMMA_SIGNALING | 62 | Up | 0.69694524 | 1.69E-07 | 0.0001337 |
| BIOCARTA_CYTOKINE_PATHWAY | 16 | Up | 1.35985937 | 3.74E-07 | 0.0001649 |
| KEGG_GRAFT_VERSUS_HOST_DISEASE | 37 | Up | 0.79390884 | 3.46E-07 | 0.0001849 |
| BIOCARTA_INFLAM_PATHWAY | 19 | UP | 1.22709045 | 7.06E-07 | 0.0002794 |
| KEGG_AUTOIMNIUNE_THYROID_DISEASE | 39 | Up | 0.72565543 | 3.30E-06 | 0.0008171 |
| KEGG_JAK_STAT_SIGNALING_PATHWAY | 112 | Up | 0.58631266 | 6.05E-06 | 0.001331 |
| REACTOME_ER_PHAGOSOME_PATHWAY | 64 | Up | 0.31851723 | 6.778-05 | 0.001412 |
| BIOCARTA_NKT_PATHWAY | 26 | Up | 0.97921623 | 1.01E-05 | 0.001737 |
| REACTOME_ANTIGEN_PRESENTATION_FOLDNG_ASSEMBLY_AND_ | 28 | Up | 0.52146072 | 9.74E-06 | 0.001737 |
| PEPTIDE_LOADING_OF_CLASS_I_MHC | | | | | |
| KEGG_ASTHMA | 17 | Up | 1.00355911 | 1.47E-05 | 0.002426 |
| REACTOME_INTERFERON_SIGNALING | 138 | Up | 0.46903952 | 1.66E-05 | 0.002625 |
| BIOCARTA_TH1TH2_PATHWAY | 16 | Up | 1.15363531 | 2.03E-05 | 0.003088 |
| KEGG_VALINE_LEUCINE_AND_ISOLEUCINE_DEGRADATION | 42 | Down | 0.33811971 | 2.13E-05 | 0.003126 |
| KEGG_FATTY_ACID_METABOLISM | 37 | Down | 0.30681536 | 2.25E-05 | 0.003182 |
| REACTOME_ANTIGEN_PROCESSING_CROSS_PRESENTATON | 75 | Up | 0.29790777 | 2.65E-05 | 0.003623 |
| KEGG_CYTOKINE_CYTOKINE_RECEPTOR_INTERACTION | 157 | Up | 0.62556163 | 3.55E-05 | 0.004603 |
| REACTOME_ENDOSOMAL_VACUOLAR_PATHWAY | 16 | Up | 0.66794746 | 4.10E-05 | 0.004978 |
| KEGG_METABOLISM_OF_XENOBIOTICS_BY_CYTOCHROME_P450 | 26 | Down | 0.61994646 | 0.000135 | 0.01215 |
| KEGG_ASCORBATE_AND_ALDARATE_METABOLISM | 12 | Down | 0.45219979 | 0.0001525 | 0.01285 |
| BIOCARTA_DC_PATHWAY | 18 | Up | 1.05610989 | 0.0001559 | 0.01286 |
| REACTOME_CYTOKINE_SIGNALING_IN_IMMUNE_SYSTEM | 236 | Up | 0.43153532 | 0.000173 | 0.0135 |
| REACTOME_HOST_INTERACTIONS_OF_HIV_FACTORS | 121 | Up | 0.27154151 | 0.0001739 | 0.0135 |
| BIOCARTA_CTL_PATHWAY | 21 | Up | 0.67928008 | 0.0002018 | 0.0148 |

TH1 as well as TH2 and TH17 cytokine expression was increased in miR1792tg cells (FIG. 8C), which argued for a master regulator pathway that is relevant to the differentiation of all of these subsets.

To identify which molecular pathways were regulated by miR-17˜92, the inventors analyzed curated gene sets enriched for differentially expressed genes between T^{1792tg/tg} and T^{1792Δ/Δ}. At 24 h, the gene sets with the highest statistical significance and largest average fold change were related to cytokines, inflammation and T cell differentiation. Next, the inventors performed enrichment analysis on DoRothEA regulons (Garcia-Alonso L. et al. Genome Res. 2019. 29(8):1363-1375) to identify transcription factors (TF) activity that could explain the regulated pathways. At 24 h, the five most significantly enriched TF regulons with the highest fold change contained two NFAT members (NFATC2, NFATC3) as well as RELA, NF-×B1 and GATA3 (FIG. 8C). Since NFAT TFs are important for T cell activation and differentiation but miR-17˜92 is not known to promote NFAT activity in T cells, the inventors focused on the calcineurin/NFAT axis. Genes belonging to regulons NFATC2 and NFATC3 include many T cell lineage-defining TFs, cytokines and cytokine receptors and most of them were highly regulated by miR-17˜92 (FIG. 8D). This confirmed that miR-17˜92 constitutes a central regulator of T cell activation and suggests that transgenic miR-17˜92 can act through canonical pathways to functionally substitute for CD28 for differentiation of TFH and Th1 in vivo (FIG. 5).

To deepen the mechanistic understanding the inventors sought to analyze direct miR-17˜92 target genes. Based on previous observations (FIG. 8A,B), the inventors focused on naïve T cells and the 24 h time point since indirect effects likely increased after this time point. To visualize the effect of individual miR-17˜92 cluster miRNAs on their target genes the inventors compared expression of genes predicted by TS and with AHC>5 reads for each miRNA seed family to all genes without any seed match for that family. As illustrated by the miR-17 seed family, the miR-17˜92 transgene repressed miR-17 target genes in naïve T cells (FIG. 8E, left panel) but absence of miR-17˜92 had no effect on the expression of miR-17 target genes (FIG. 8E, right panel). In contrast, after T cell activation miR-17 target genes were repressed in T^{1792tg/tg} T cells (FIG. 8F, left panel) and derepressed in T^{1792Δ/Δ} T cells (FIG. 8F, right panel). Similar effects were observed for all seed families although to a lesser extent for the miR-18 seed family. Finally, the inventors defined genes as empirically validated miR-17˜92 targets if they fulfilled the following criteria: i) significant derepression in T^{1792Δ/Δ} vs wt and significant repression in T^{1792tg/tg} vs wt at 24 h ii) predicted TS match iii) >5 AHC reads and iv) posttranscriptional regulation based on EISA. Applying these criteria across the 4 seed families from miR-17˜92 defined a set of empirically supported direct miR-17˜92 target genes (Table 1). These genes included previously described miR-17˜92 targets validated in T cells, such as Phlpp2 (Kang S. G. et al. Nat Immunol. 2013. 14(8):849-57) and Cyld, validated in B cells, confirming the validity of the analysis (Jin H. Y. et al. EMBO J. 2013. 32(17):2377-91). In addition, this approach identified many less studied genes as miR-17˜92 targets in T cells. Notably, several of these genes are negative regulators and some are known or suspected tumor suppressors in various cell types.

In summary, the inventors stringently defined a set of miR-17˜92 target genes in T cells. miR-17˜92 did not repress target genes in naïve T cells but transgenic miR-17˜92 did. However, this repression did not substantially affect the entire transcriptome in naïve T cells. In contrast, after T cell activation miR-17˜92 directly repressed target genes which had important consequences enhancing major T cell signaling pathways. Thus, miR-17˜92-mediated gene repression is very powerful to shape the T cell transcriptome during T cell activation. Its calcineurin/NFAT enhancing activity likely contributes to the phenotypic rescue of CD28^{-/-} T cells by transgenic miR-17˜92.

2.6 miR-17˜92 Targets are Upregulated in CD28ko Cells and Repressed to Wt Levels by Transgenic miR-17˜92 Expression

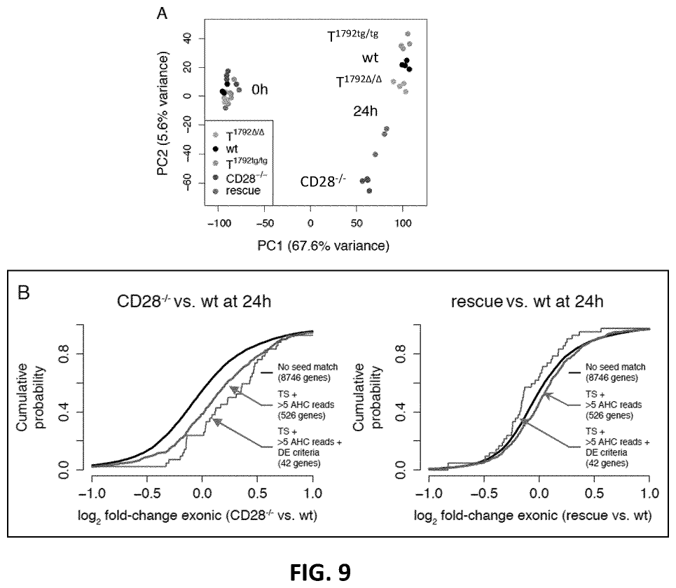

Following hypothesis of the inventors, they isolated RNA from naïve and 24 h activated CD4 T cells from CD28ko, wt, rescue, miR1792tg and miR1792lox mice to investigate if the gene signature in CD28ko cells is rescued by the transgenic expression of miR-17˜92. Unbiased PCA analysis separated time points on PC1. Gene expression was overlapping in naïve samples, and PC2 split up the activated samples in genotypes with the CD28ko samples being the most different from the others. Rescue cells were more similar to the other samples, and miR1792tg samples were the most different to CD28ko samples (FIG. 9A). This argues that only parts of the gene expression that is different between CD28ko and wt samples can be rescued by forced miR-17˜92 expression. The inventors then analysed the dataset looking at different subset of genes: they compared the ratio of the log 2 (fold change) to the cumulative fraction of all log 2 fold changes in different contrasts. They further display the set of genes in their dataset that have a binding site for miR17 (or any other member of the miR-17˜92, data not shown) in light grey and see that this gene sets is shifted to the right in the comparison miR1792lox vs. wt (FIG. 9B), which indicates that the sum of all genes that have a binding site for miR-17 is actually increased in cells that do not express the cluster. The same set of genes is shifted to the left, so lower expression, in the comparison miR1792tg to wt (data not shown). Furthermore, they focused on the subset of genes that is increased in expression in miR1792lox, decreased in miR1792tg, contain a binding site for miR-17 according to targets can and are only changed at exonic, but not intronic level. Similar to the shift towards increased gene expression of genes with miR17 binding site in miR1792lox vs. wt (data not shown), the gene expression was also shifted to higher expression in CD28ko vs. wt (FIG. 9C), which suggests that genes that are regulated by miR-17˜92 are also regulated by CD28. This shift was completely abolished with transgenic expression of miR-17˜92 expression in rescue cells (FIG. 9B), which indicates that some dysregulation seen in CD28ko cells might be explained by lacking miR-17˜92, and therefore their expression can be regulated by transgenic miR-17˜92 expression. This was true for miR-17 targeted genes but also for the other members of the miR-17˜92 cluster (data not shown).

2.7 Rcan3 3′UTR is Targeted by miR-17 and Expression Level is Dependent on CD28 or miR-17˜92 Expression

The second RNA sequencing dataset showed a ˜0.5 fold increase of Rcan3 RNA in CD28ko cells in comparison to wt, which is similar to miR1792lox vs. wt cells. This was reduced to wt levels in rescue cells. Protein expression of Rcan3 was 2-fold increased in CD28ko and miR1792lox cells in comparison to wt, which was reduced to wt by transgenic miR-17˜92 expression in the rescue cells. Nonetheless, even if the RNA expression level in miR1792tg cells was decreased in comparison to wt cells, the protein level was actually not significantly changed. This argues that the intrinsic miR-17˜92 expression levels reached in wt cells might be sufficient for the regulation of Rcan3 during activation.

2.8 Sensitivity to Cyclosporin a is Influenced by CD28 and Rescued by Transgenic miR-17˜92 Expression

As mentioned in previous sections, the data indicated that all T helper subsets are affected by loss of miR-17˜92 or CD28 signaling, and there is a rescue effect in different subsets as well. One pathway that could explain for this phenotype is the Calcineurin-NFAT axis. The inventors therefore mined the list of potential target genes (Table 1) for candidates that might negatively regulate this pathway, thereby increasing the signalling of the NFAT pathway if their expression is reduced. One candidate for this is Regulator of calcineurin 3 (Rcan3), which was reported to interact with and inhibit Calcineurin (Mulero, M. C., et al. Biochim Biophys Acta, 2007. 1773(3):330-41).

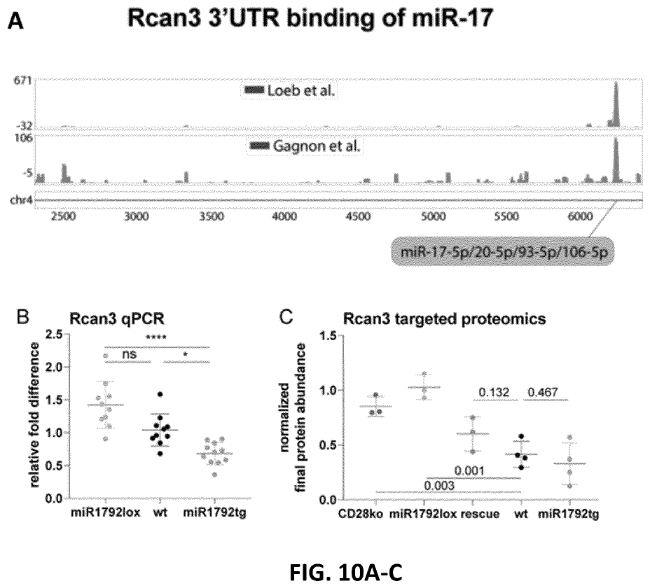

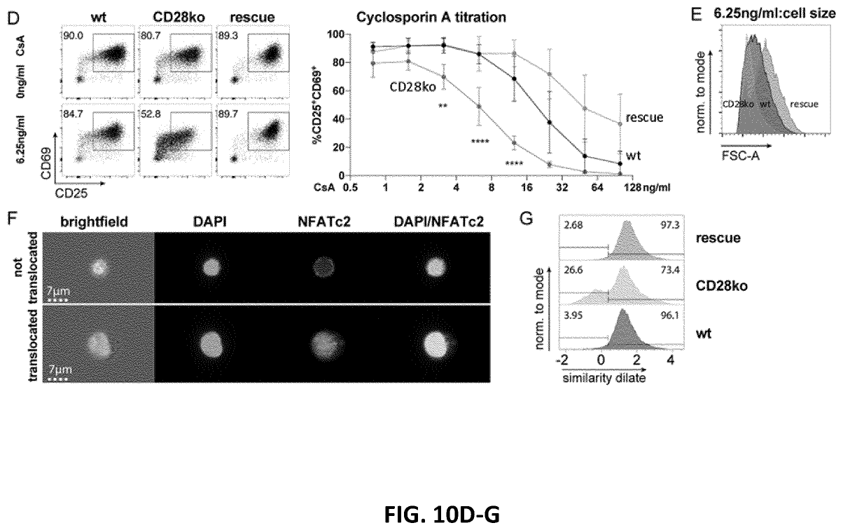

The HITSCLIP data showed that the miR-17 binding site in the 3′UTR of Rcan3 was actually bound to Argonaute at the moment of sequencing (FIG. 10A), which increased the probability that this is a true target gene. Rcan3 mRNA expression was increased in miR1792lox cells (FIG. 10B) as compared to wt, and decreased expression in miR1792tg cells. Also the protein expression was elevated in CD28ko and miR1792lox cells as compared to wt, and rescue cells had a lower Rcan3 protein expression. Furthermore, the 3′UTR of Rcan3 contains a conserved miR-17-5p 8mer binding site (chr4:135416232-135416240) and AHC confirmed a single discrete peak at this site, evidence for a direct interaction of miR-17-5p with the Rcan3 3′UTR in primary T cells (FIG. 10A and Loeb. G. B. et al. Molecular Cell. 2012 and Gagnon J. D. et al. Cell Rep. 2019. 28(8):2169-2181).

The inventors then hypothesized that increased Rcan3 expression might increase the sensitivity of the cells to calcineurin inhibitor Cylcosporin A (CsA), and therefore activated the cells in the presence of increasing concentrations of CsA. As shown in FIG. 3, all samples started from similar percentages of CD25⁺CD69⁺ expression, but CD28ko cells reacted more sensitively to increasing CsA concentrations (FIG. 10D) while rescue cells were even more resistant. This was also true when looking at cell size, shown as FSC-A (FIG. 10E). Normally, Calcineurin dephosphorylates NFAT during T cell activation, and only then NFAT can translocate to the nucleus and initiate important transcriptional programs. With the inhibition of Calcineurin, this translocation is reduced. The inventors show this in FIG. 10F using the imagestream technique. They activated cells in the presence of low dose cyclosporin (6.25 ng/ml, which was shown in 9d that CD28ko react but not wt or rescue cells) and looked at the translocation of NFAT. Only the CD28ko sample showed a population of cells that had a lower similarity dilate (FIG. 10G), which is indicative of less translocation.

2.9 Sensitivity to Calcineurin Inhibitors (e.g. Cyclosporin a, FK506) in Wildtype Cells can be Reduced by Transgenic Expression of miR-17˜92 or Deletion of miR-17˜92 Target Genes, e.g. Rcan3

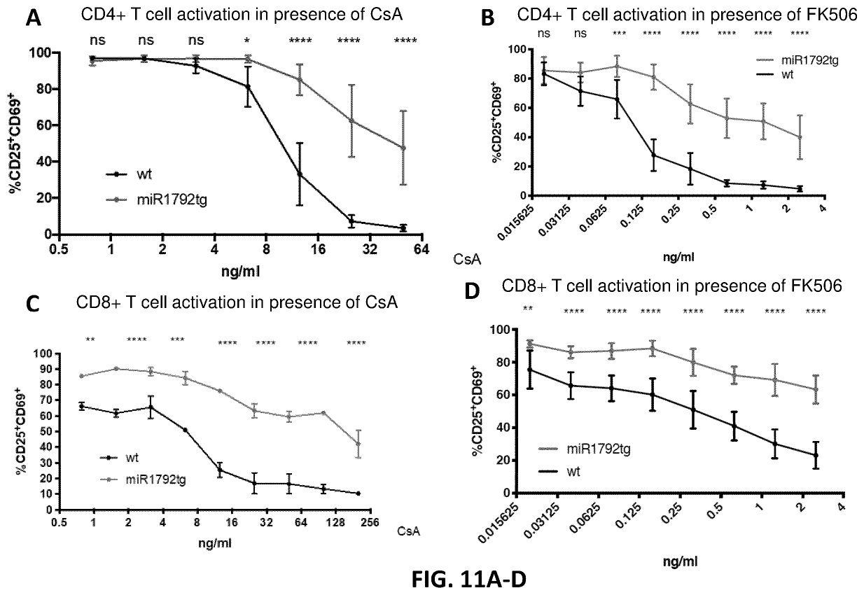

Since the higher sensitivity of CD28ko cells to cyclosporin A could be reverted by the transgenic expression of miR-17˜92, the inventors hypothesized that transgenic expression of miR-17˜92 generally could increase the resistance to this immunosuppressant even in CD28-sufficient cells. They therefore activated CD4+ T cells and CD8+ from wildtype and miR1792tg cells in the presence of increasing concentrations of calcineurin inhibitors such as cyclosporin A (FIG. 11A,C) and FK506 (FIG. 11B,D) and found that indeed transgenic miR-17˜92 expression in cells CD4+ and CD8+ T cells with normal co-stimulation leads to increased resistance to cyclosporin A and FK506 (FIG. 11).

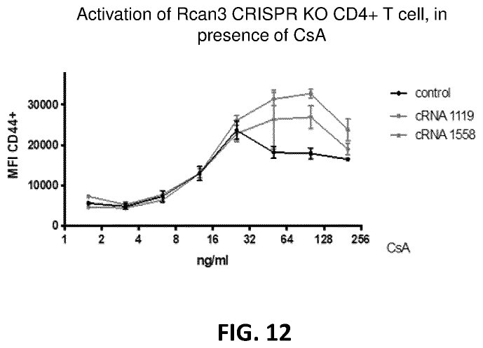

As the previous data suggest that CD28 costimulation engages miR17-5p to repress Rcan3, the inventors hypothesized that repression or elimination of Rcan3 expression could increase the resistance to calcineurin inhibitor in T cells, even without altering miR-17˜92 expression. CD4 T cells were electroporated with control gRNA or 2 different gRNAs targeting Rcan3 (GAGAAATACGAACTGCACGC; crRNA 1119, SEQ ID NO: 47 and GATGGTCTTCGGTGAAAATG, crRNA 1558, SEQ ID NO: 48). Cells were rested in vitro, then reactivated in the presence of various CsA concentrations. As expected, CD44 upregulation (marker of T cell activation) was inhibited at higher CsA concentrations in wildtype cells. In contrast, CRISPR/Cas9-mediated deletion or Rcan3 rendered Rcan3 KO cells resistant to CsA (FIG. 12). Thus, deleting a miR-17˜92 target gene conveyed the same therapeutic benefit as overexpressing miR-17˜92.

1. A method for adoptive cell transfer therapy in a subject in need thereof comprising administering to the subject a therapeutically effective amount of a calcineurin inhibitor (CNI)-resistant immune cell in which regulatory activity of the miR-17˜92 cluster or paralogs thereof is increased wherein said CNI-resistant immune cell is administered in combination with a CNI in said subject.

2. The method according to claim 1 wherein said CNI-resistant immune cell is engineered to overexpress at least one miRNA selected from the group consisting of miR-17, miR-18a, miR-19a, miR-20a, miR-19b-1 and miR-92a-1, miR106a, miR-18b, miR-19b-2, miR-20b, miR-92a-2, miR-363, miR-106b, miR-93, miR-25, preferably miR-17 er and miR19.

3. The method according to claim 2 wherein said CNI-resistant immune cell is engineered by introducing into said CNI-resistant immune cell a nucleic acid construct comprising at least one miRNA sequence selected from the group consisting of: SEQ ID NO: 17 to 46.

4. The method according to claim 2 wherein said nucleic acid construct comprises at least one pre-miRNA sequence selected from the group consisting of: SEQ ID NO: 1 to 16.

5. The method according to claim 3 wherein said nucleic acid construct is introduced into said CNI-resistant immune cell by electroporation.

6. The method according to claim 1 wherein said CNI-resistant immune cell is engineered to inactivate the expression of at least one miR-1792 cluster target gene.

7. The method according to claim 6 wherein said CNI-resistant immune cell is engineered to introduce a Cas9/CRISPR complex able to target an Rcan3 gene into said CNI-resistant immune cell.

8. The method according to claim 1, wherein said calcineurin inhibitor is selected from the group consisting of: cyclosporine A, FK506 and CTLA-4 Ig.

9. The method according to claim 1, wherein said CNI-resistant immune cell is selected from the group consisting of: a T cell, a B cell, a Tumor infiltrating lymphocyte, NK cell, a macrophage and a regulatory T cell.

10. The method according to claim 9 wherein said CNI-resistant immune cell originates from said subject or a donor.

11. The method according to claim 1, wherein said CNI-resistant immune cell further express a recombinant antigen receptor.

12. A method of treating a cancer, an autoimmune disease, an inflammatory disease, an infectious disease, a disease requiring hematopoietic stem cells transplantation (HSCT) or organ rejection in a subject in need thereof, comprising administering to the subject a therapeutically effective amount of a calcineurin inhibitor (CNI)-resistant immune cell in which regulatory activity of the miR-17˜92 cluster or paralogs thereof is increased, and wherein said (CNI)-resistant immune cell is administered to the subject in combination with a CNI.

13. A pharmaceutical composition comprising a CNI-resistant immune cell in which regulatory activity of the miR-17˜92 cluster or paralogs thereof is increased, a calcineurin inhibitor and a pharmaceutically acceptable carrier.

14. The A method for adoptive cell transfer therapy in a subject in need thereof comprising administering to the subject a pharmaceutical composition comprising a CNI-resistant immune cell in which regulatory activity of the miR-17˜92 cluster or paralogs thereof is increased, a calcineurin inhibitor and a pharmaceutically acceptable carrier.

15. The method according to claim 6, wherein the at least one miR-1792 cluster target gene is an Rcan3 gene.

16. The method according to claim 11 wherein the recombinant antigen receptor is a chimeric antigen receptor.

17. The method of claim 12, wherein the disease that is treated is selected from the group consisting of: graft versus host disease, hematologic malignancy, posttransplant lymphoproliferative disease and an autoimmune disease.
